(19) United States
(12) Patent Application Publication
(10) Pub. No.: US 2017/0175169 A1
(43) Pub. Date: Jun. 22, 2017

(54) CLINICAL DECISION SUPPORT SYSTEM UTILIZING DEEP NEURAL NETWORKS FOR DIAGNOSIS OF CHRONIC DISEASES

(71) Applicant: Min LEE, Albany, CA (US)

(72) Inventors: Min LEE, Albany, CA (US); Seok Yong MOON, GYUNGGIDO (KR); Matthew LEE, Albany, CA (US); Nupur RAYCHAUDHURI, Ann Arbor, MI (US); Saroj Kumar BASAK, Panorama City, CA (US); Phillip Scott ALEXANDER, Indianapolis, IN (US)

(21) Appl. No.: 15/148,198

(22) Filed: May 6, 2016

Related U.S. Application Data
(60) Provisional application No. 62/269,858, filed on Dec. 18, 2015.

(51) Int. Cl. C12Q 1/68; G06F 19/00; G01N 33/543

(52) U.S. Cl. CPC C12Q 1/6804; G01N 33/54373; G06F 19/345

(57) ABSTRACT

The inventors have developed a clinical decision support system (“CDSS”) and associated devices to diagnose chronic diseases in patients using a large number of biomarkers. The inventors have utilized a process for identifying thousands of biomarkers that could be relevant to potential diseases. Once these biomarkers are identified, the ones that have the most affinity for relevant biomarkers are retained. Then, a clinical decision support system can utilize the thousands of biomarkers, and apply a DNN based machine learning algorithm to diagnose chronic diseases.

### CROSS-REFERENCE TO RELATED APPLICATIONS

The application claims priority under 35 U.S.C. §119 to U.S. Provisional Application Ser. No. 62/269,858, entitled CLINICAL DECISION SUPPORT SYSTEM UTILIZING DEEP NEURAL NETWORKS FOR DIAGNOSIS OF CHRONIC DISEASES, filed Dec. 18, 2015, the disclosure of which is incorporated in its entirety by this reference.

### FIELD

The present invention is directed to clinical decision support systems and associated biochips for diagnosing chronic diseases.

### BACKGROUND

The following description includes information that may be useful in understanding the present invention. It is not an admission that any of the information provided herein is prior art or relevant to the presently claimed invention, or that any publication specifically or implicitly referenced is prior art.

Chronic diseases are diseases that are mostly non-communicable (cannot be based from person to person) and according to the definition by the U.S. National Center for Health Statistics, are any disease that lasts for three months or longer. Chronic diseases include cancer, cardiovascular diseases (e.g. heart attack and stroke), chronic respiratory diseases, diabetes, lupus, and strokes. Chronic diseases are accountable for 70% of all death in the US and by far the leading cause of death in the world. For example, in 2002, the leading chronic diseases—cardiovascular disease, cancer, chronic respiratory disease, and diabetes—caused 29 million deaths worldwide.

In spite of this, adequate screening or even diagnostic methods are not available for detecting most chronic diseases at the earliest stages when they are most treatable. For example, many cancers are not detected into later stages when treatment is much more difficult and outcomes are much less favorable. As one example, lung cancer screening is currently performed with a scan, which may cost health care systems around $500 per scan and has a false positive rate of about 20%.

Recently, some Clinical Decision Support Systems (“CDSS”) or health information technology systems that assist caregivers in making diagnostic, treatment and other decisions, have developed the ability to screen for some diseases by analyzing concentrations of certain biomolecules or (“biomarkers”) in the body and other information. The CDSS can analyze the input information (e.g., concentrations of the biomarkers that are known to be associated with a particular chronic disease), and provide some information on the likelihood a particular patient has the disease.

Current CDSS rely on certain statistical methods and machine learning programs (computer program algorithms used in decision making processes) to compare a patient's tested input data (e.g. biomarker concentrations) with data from previously tested subjects with known disease status (e.g.,] biomarker concentration data from test subjects that are known to have a particular disease and controls without the disease. If the patient's biomarker concentration data matches closer to the disease sample after comparison, the CDSS may determine that patient likely has that particular disease.

To date, CDSS have implemented four different types of machine learning programs for attempting to diagnose disease including: (1) decision trees (“DT”), (2) Bayesian networks (“BN”), (3) artificial neural network (“ANN”), or (4) support vector machines (“SVM”). Most CDSS commonly implement DT programs because of their simplicity and ease of understanding. DT are classification graphs that match input data to questions asked at each consecutive step in a decision tree. The DT program moves down the “branches” of the tree based on the answers to the questions (e.g., First branch: Is the patient male? yes or no. Branch two: Is the patient having trouble urinating? yes or no. etc.). DT have been employed for solving various biological problems. These include diagnostic error analysis, finding potential biomarkers, and proteomic mass spectra classification. [9, 10, 11 12]. FIG. 16 is an example of a decision tree used in speech diagnosis [23].

Bayesian networks (“BN”) are based on likelihood something is true based on given independent variables and are modeled based on probabilistic relationships. For example, FIG. 17 is a simple BN based on the relationship between a rain, activation of a sprinkler and whether grass is wet.

BN are based purely on probabilistic relationships that determine the likelihood of one one variable based on another or others. For example, BN can model the relationships between symptoms and diseases. Particularly, if a patient's symptoms or biomarkers levels are known, a BN can be used to compute the probability that a patient has a particular disease. Thus, using an efficient BN algorithm, an inference can be made based on the input data. They are commonly used by the medical domain to represent reasoning under uncertain conditions for a wide range of applications, including disease diagnostics, genetic counseling, and emergency medical decision support system (MDSS) design. [13, 14, 15]

Artificial neural networks (“ANN”) are computational models inspired by an animal's central nervous system. They map inputs to outputs through a network of nodes. However, unlike BN, in ANN the nodes do not necessarily represent any actual variable. Accordingly, ANN may have a hidden layer of nodes that are not represented by a known variable to an observer as illustrated in FIG. 18.

ANNs are capable of pattern recognition and have been used for the medical and diagnostics fields. Their computing methods make it easier to understand a complex and unclear process that might go on during diagnosis of an illness based on input data a variety of input data including symptoms. While still facing steep limitations, ANN has demonstrated to be suitable in CDSS design and other biomedical applications, such as diagnosis of myocardial infarction, MDSS for leukemia management, and cancer detection. [16, 17, 18]

Support vector machines (“SVM”) came about from a framework utilizing of machine learning statistics and vector spaces (linear algebra concept that signifies the number of dimensions in linear space) equipped with some kind of limit-related structure. In some cases, they may determine a new coordinate system that easily separates inputs into two classifications. For example, a SVM could identify a line that separates two sets of points originating from different classifications of events as illustrated in FIG. 19.

In this example from Wikipedia, H3 clearly separates the two sets of points, while H1 and H2 do not. SVM have become an increasing interest to biomedical researchers. They have been applied practically and are theoretically well-founded, but can sometimes be difficult to understand. SVMs have been applied to a number of biological domains, such as MDSS for the diagnosis of tuberculosis infection, tumor classification, and biomarker discovery. [19, 20, 21].

### SUMMARY

### Conventional CDSS Systems Implementing Machine Learning Algorithms

Clinical Decision Support Systems (“CDSS”) are systems that generate case-specific advice using active knowledge systems and two or more items of patient data. [1] As discussed above, the majority of CDSS available today use one or more of four types of algorithms, decision trees (DT), Bayesian networks (BN), artificial neural networks (ANN), and support vector machines (SVM). Following are specific examples of systems utilizing machine learning algorithms to screen or diagnose diseases, all with significant and practical application limiting shortfalls.

For example, Won et al., discloses a CDSS that relies on the use of DT to distinguish between Renal Cell Carcinoma (RCC) and other urological diseases. However, Won's platform identified only five proteins from serum as biomarkers input into the platform. [2]

According to the publication by Jiang et al. in the journal of Cancer Information, 2014, their CDSS utilized a BN-based EBMC (efficient Bayesian multivariate classification) algorithm to predict the survivability of breast cancer. They claim their CDSS had 90% accuracy of prediction. However, this model uses many variables that are subjective and not very accurate including many invasive methods including “grade of disease”, “histological” analysis of the tumor, and is not for detection of breast cancer but for evaluation of the tumor for probability of survivability. [4]

The diagnosis of heart disease commonly utilizes various data mining techniques such as DT, ANN, BN, SVN, kernel density, multilayer perceptron (MLP), back propagation (BP) and the bagging algorithm. [5, 6, 7]. One example of an MLP CDSS system utilizes ANN-MLP, and BP to make its predictions. This system is used to predict whether a patient has any type of heart disease or not. The advantages to this system are: (1) it does not require a large amount of statistical training, (2) it is great at implicitly detecting complex nonlinear relationships associated between independent and dependent variables, and (3) its ability to detect interactions between the predictor variables. This system can also utilize many other different algorithms for training the system. Disadvantages include: (1) it is very prone to overfitting the data, (2) has a greater computational burden than most systems, (3) suffers from its “black box” nature, and the empirical nature of the model development. [8]

The CANFIS-GA system uses ANN, Neuro-Fuzzy, and a genetic algorithm. It is used to predict and categorize four different types of heart disease. The survey of the system utilized 303 cases of heart disease. This system has a high accuracy, performance and generalization ability. However, it also relies on subjective metrics from patients including chest pain, etc. Furthermore, it has only an intermediate level for reliability and it is not very easy to interpret the results. [8]

The AptaCDSS-E system utilizes four different algorithms to make its predictions for cardiovascular disease (CVD) using biomarkers. They are SVM, ANN, DT, and BN. The advantages to this system are it is cost effective, and it is easy to interpret. However, it has a poor ability at generalization, only mediocre accuracy and reliability, tends to have biased decision and classification results and a high risk of overfitting classifiers due to extra learning. [8]

The IHDPS system utilizes DT, ANN and Naïve Bayes (NB) to make predictions on generalized heart disease. Each prediction was based on only one of these algorithms. The study used 909 data from the Cleveland heart disease database with 15 medical characteristics. The training set included 455 of these data and the testing set included 454 data. It was understood that the BN performed the best followed by ANN and finally DT. While this system is very easy to interpret the results, it has a mediocre accuracy and reliability. It has a very low quality of generalization ability. [8]

The IEHAPS system uses K-means, ANN and maximum frequent item sets (MAFIA) algorithms to predict a patient's risk of heart disease. The advantages are its ease of interpretation and accuracy. Its disadvantage is the overall cost. [8]

DT-Fuzzy system involves DT and the fuzzy algorithm methods to determine the risk of heart disease. It is very easy to interpret the results, with a mediocre accuracy and overall cost. It is not very reliable. [8]

CDSS platforms to screen and diagnosis for chronic diseases based on the above described algorithms do not generate highly accurate results and end up producing high false positive and high false negative results. These are due to incompetency of the corresponding biomarkers selection to the chronic diseases and major limitations of the algorithms used for the artificial intelligence machine learning process.

### Overview

Since the current available CDSS platforms have major limitations for producing highly accurate screening and diagnostic results, the inventors have developed novel systems and methods to implement a DNN based algorithm to evaluate a relatively large number of biomarkers to generate highly accurate diagnosis of chronic diseases.

Accordingly, while some CDSS have applied machine learning algorithms to diagnose some chronic diseases, none have been effective and have been able to reliably diagnose many chronic diseases. Furthermore, no practical and robust system exists for reliably analyzing patient data to diagnose and screen chronic diseases. For instance, many of the CDSS (as discussed above) utilize subject information including symptoms, or a limited set of biomarkers with low specificity or sensitivity.

One of the main problems for diagnosing chronic diseases using biomarkers is that generally, most chronic diseases manifest as a malfunction of the patient's natural biochemistry and therefore involves detect minute different amounts of a many different biomarkers. This includes generally the vast amount of proteins and other biomolecules that interact to drive the body's internal processes. In contrast, screening tools for infectious diseases (e.g. HIV), can usually target a single or handful of biomarkers (i.e. the HIV virus).

Accordingly, the biomarkers that could be relevant for any particular chronic disease is extraordinarily vast, and may be different depending on (1) the characteristics of the patient, (2) the stage of the disease, (3) changing factors within the patient, and (4) others. Therefore, the amount of information that may be relevant using biomarkers has been the vast number of biomarkers and complex interactions. This is likely why, to date, clinical research has focused on biomarkers that have been validated biologically as a cause or pathway related to the actual chronic diseases. This has allowed researchers to look at a smaller number of biomarkers (e.g. 35) that may be most related to the disease.

However, because most biomarkers can still be associated with different types of diseases, this approach has largely failed to create a practical, robust, and effective diagnostic CDSS tool for the majority of chronic disease. Therefore, the majority of chronic diseases remain undiagnosed, and result in widespread death and suffering due to their lack or tardiness of detection.

This devastating fact is the main driving force which has pushed the inventors to develop the disclosed CDSS platform that can screen and diagnose a multitude of chronic diseases so that patients can start treatments as early as possible to maximize their outcomes against the deadly diseases. With the development of the CDSS Platform disclosed here, screening and diagnosis of chronic diseases will become relatively non-invasive.

Despite these challenges, the inventors have discovered: (1) a reliable way to detect the concentrations of a large number of the most relevant biomarkers for diagnosing particular diseases, (2) a reliable way to detect these biomarkers taken relatively non-invasively and (3) a DNN based classification machine learning platform that can reliably interpret the vast amount of complex biomarker data output from the system to reliably diagnose chronic diseases.

At least three steps may be important to develop this screening and diagnosis solution: (1) identify a large number known and unknown biomarkers that can be reliably detected, (2) take blood, urine, saliva or any combination of these samples from patients with known disease status and test them for biomarker concentrations, and (3) using this data from concentration of biomarkers to train or develop a DNN based machine learning program for diagnosing diseases.

To begin with, inventors have developed technologies to identify many known and unknown biomarkers corresponding to chronic diseases from human samples such as blood, saliva, and urine, or any combinations of the human samples. The total numbers of selected biomarkers from human samples used could be more than 1 or less than 10,000. For instance, 100 to 10,000, or 500 to 1,000 or other ranges.

In the second step, blood, urine, and saliva, or any combination of these human samples will be taken from many human subjects for each target disease. The blood, urine, and saliva samples, or any combination of these human samples will be collected from the human subject, will be tested for the concentration of biomarkers in each of the subjects from each of the diseases. This reaction data from each human subject will be used as the input to train or “teach” the disclosed CDSS platform to diagnose diseases.

The reaction data input into the CDSS will comprise of data from samples known to be positive for a chronic disease (abnormal, diseased) data and negative for a chronic disease (normal, control) data. For example, 40% of total data can be used as positive (abnormal) data and 40% of total data can be used as negative (normal) data to train the CDSS platform. The remaining 20% of the data could be used as testing data during the training stages as performance validation data for better generalization by the DNN.

Finally, the advanced artificial intelligence machine learning technology of DNN algorithms can be used and trained using the input data generated so that this advanced CDSS platform can screen and diagnosis chronic diseases as accurately as possible. The more input data fed into the CDSS Platform during training, the higher the accuracy of screening and diagnostic results.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts, in accordance with various embodiments of the present invention, an overview of a system for implementing a CDSS platform for diagnosing chronic diseases;

FIG. 2 depicts, in accordance with various embodiments of the present invention, a flow chart illustrating an overview of the process for diagnosing a disease using the disclosed systems and methods;

FIG. 3 depicts, in accordance with various embodiments of the present invention, a flow chart illustrating a method for identifying aptamers to construct an array;

FIG. 3 depicts, in accordance with various embodiments of the present invention, an array;

FIG. 4 depicts, in accordance with various embodiments of the present invention, a flow chart illustrating a method for training a DNN based algorithm by inputting data detected from human sample reactions with an array;

FIG. 5 depicts, in accordance with various embodiments of the present invention, a flow chart illustrating an example of a method for outputting a diagnosis using a an array after reaction with a human sample;

FIG. 6 depicts, in accordance with various embodiments of the present invention, an array after reaction;

FIG. 7 depicts, in accordance with various embodiments of the present invention, a schematic representation of a DNN based algorithm;

FIG. 8 depicts, in accordance with various embodiments of the present invention, a flow chart illustrating a training process for an RBM based DNN algorithm;

FIG. 9 depicts, in accordance with various embodiments of the present invention, the structure of the RBM algorithm used to train each hidden layer of a DNN based algorithm;

FIG. 10 depicts, in accordance with various embodiments of the present invention, a schematic representation of a contrast divergence technique;

FIG. 11 depicts, in accordance with various embodiments of the present invention, a flow chart illustrating a training procedure for a stacked RBM based DNN algorithm;

FIG. 12 depicts, in accordance with various embodiments of the present invention, a flow chart illustrating a training procedure for a stacked RBM based DNN algorithm;

FIG. 13 depicts, in accordance with various embodiments of the present invention, a graphical representation of gradient descent;

FIG. 14 depicts, in accordance with various embodiments of the present invention, a schematic representation of a CNN based DNN algorithm

FIG. 15 depicts, in accordance with various embodiments of the present invention, a flow chart illustrating a process for combining outputs of a RBM and CNN based DNN algorithm

FIG. 16 depicts a prior art machine learning algorithm;

FIG. 17 depicts a prior art machine learning algorithm;

FIG. 18 depicts a prior art machine learning algorithm;

FIG. 19 depicts a prior art machine learning algorithm;

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a sub combination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be implemented.

### SELECTED DEFINITIONS

The term “sample” or “biological sample” as used herein denotes a sample taken or isolated from a biological organism, e.g., a tumor sample from a subject. Exemplary biological samples include, but are not limited to, cheek swab; mucus; whole blood, blood, serum; plasma; urine; saliva; semen; lymph; fecal extract; sputum; other body fluid or biofluid; cell sample; tissue sample; tumor sample; and/or tumor biopsy etc. The term also includes a mixture of the above-mentioned samples. The term “sample” also includes untreated or pretreated (or pre-processed) biological samples. In some embodiments, a sample can comprise no cells from the subject. In other embodiments, a sample can comprise one or more cells from the subject. In some embodiments, a sample can be a tumor cell sample, e.g. the sample can comprise cancerous cells, cells from a tumor, and/or a tumor biopsy.

In various embodiments of the present invention, a chronic disease includes but is not limited to cancer, cardiovascular diseases, chronic respiratory diseases, diabetes, lupus, and stroke.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant tumors, as well as dormant tumors or micrometastasis. Cancers which migrate from their original location and seed vital organs can eventually lead to the death of the subject through the functional deterioration of the affected organs. As used herein, the term “invasive” refers to the ability to infiltrate and destroy surrounding tissue. Melanoma is an invasive form of skin tumor. As used herein, the term “carcinoma” refers to a cancer arising from epithelial cells. Examples of cancer include, but are not limited to, nervous system tumor, brain tumor, nerve sheath tumor, breast cancer, colorectal cancer, colon cancer, rectal cancer, bowel cancer, carcinoma, lung cancer, hepatocellular cancer, gastric cancer, pancreatic cancer, cervical cancer, ovarian cancer, liver cancer, bladder cancer, cancer of the urinary tract, thyroid cancer, renal cancer, renal cell carcinoma, carcinoma, melanoma, head and neck cancer, brain cancer, and prostate cancer, including but not limited to androgen-dependent prostate cancer and androgen-independent prostate cancer. Examples of brain tumor include, but are not limited to, benign brain tumor, malignant brain tumor, primary brain tumor, secondary brain tumor, metastatic brain tumor, glioma, glioblastoma, glioblastoma multiforme (GBM), medulloblastoma, ependymoma, astrocytoma, pilocytic astrocytoma, oligodendroglioma, brainstem glioma, optic nerve glioma, mixed glioma such as oligoastrocytoma, low-grade glioma, high-grade glioma, supratentorial glioma, infratentorial glioma, pontine glioma, meningioma, pituitary adenoma, and nerve sheath tumor. Nervous system tumor or nervous system neoplasm refers to any tumor affecting the nervous system. A nervous system tumor can be a tumor in the central nervous system (CNS), in the peripheral nervous system (PNS), or in both CNS and PNS. Examples of nervous system tumor include but are not limited to brain tumor, nerve sheath tumor, and optic nerve glioma.

### Overview

The inventors have developed a clinical decision support system (“CDSS”) and associated devices to diagnose chronic diseases in patients using a large number of biomarkers. The inventors have utilized a process for identifying thousands of biomarkers that could be relevant to potential diseases. Once these biomarkers are identified, the ones that have the most affinity for relevant biomarkers are retained. Then, a clinical decision support system can utilize the thousands of biomarkers, and apply a DNN based machine learning algorithm to diagnose chronic diseases.

Generally, patients are filled with information in the form of the biomolecules dispersed throughout their body. For instance, proteins created by transcription and translation of the a patient's DNA (i.e. gene expression), include long chains of amino acids that are responsible for the vast majority of the enzymes, building blocks of most of the body's structures, and drive most of the body's processes.

Most chronic diseases involve a patient's biochemistry malfunctioning in some manner, with many of the normal processes and body structures disrupted, augmented, down-regulated, etc. Accordingly, one of the processes that may be disrupted is gene expression, or the amount and type of proteins created from a patient's DNA. For instance, the process of metastasis involves an intricate interplay between altered cell adhesion, survival, proteolysis, migration, and lymph-/angiogenesis.

Accordingly, the biochemical messages in the body that are the fingerprint of cancer and other chronic diseases are primarily an extraordinary complex array of every changing protein and nucleic acid chains in the body. These protein or biomolecules in the body are also known as “biomarkers” due to fact that there existence and more importantly their concentrations in combination with other biomarkers may indicate the existence of particular diseases. Other biomarkers exist in the body, including short strains of amino acids, viruses, tissues, cells, enzymes (many of which are comprised of proteins/amino acids) and others biomolecules.

Researchers have discovered short chains of single stranded DNA or RNA named “aptamers” can bind to biomarkers with high specificity and affinity. In other words, these single stranded molecules, each with different amino acid sequences, each bind exclusively to unique biomarkers (e.g. enzymes, proteins). Accordingly, these aptamers can be utilized to identify which proteins and other biomarkers are expressed in a patient. Furthermore, if the biomarkers (and aptamers that bind to them) responsible for a particular disease can be discovered, the aptamers can be utilized to test for these biomarkers and thus infer whether they have a disease. However, existing research has generally only discovered, for each disease, a small number of known biomarkers where a link between the biomarker or handful of biomarkers and the disease can be established analytically, so the causal or direct relationship can mapped in a biochemical pathway.

However, for many chronic diseases, thousands of biomarkers interplay at varying concentrations, that are different from a healthy individual, and relatively small differences in these biomarkers may indicate an individual has a disease. Furthermore, personal variations in these biomarkers have made existing algorithms for diagnosing diseases based on a small number of biomarkers relatively inaccurate. Furthermore, adequate algorithms and methods for identifying known and unknown biomarkers for these diseases have not been developed.

Despite these challenges, the inventors have discovered: (1) a reliable way to identify a large number of relevant aptamers (and associated biomarkers) for diagnosing particular diseases, (2) a reliable way to detect these biomarkers using aptamers based on blood, urine and/or saliva samples taken from a patient and (3) a DNN based classification machine learning platform that can reliably interpret the vast amount of complex biomarker data output from these detection systems to reliably diagnose chronic diseases.

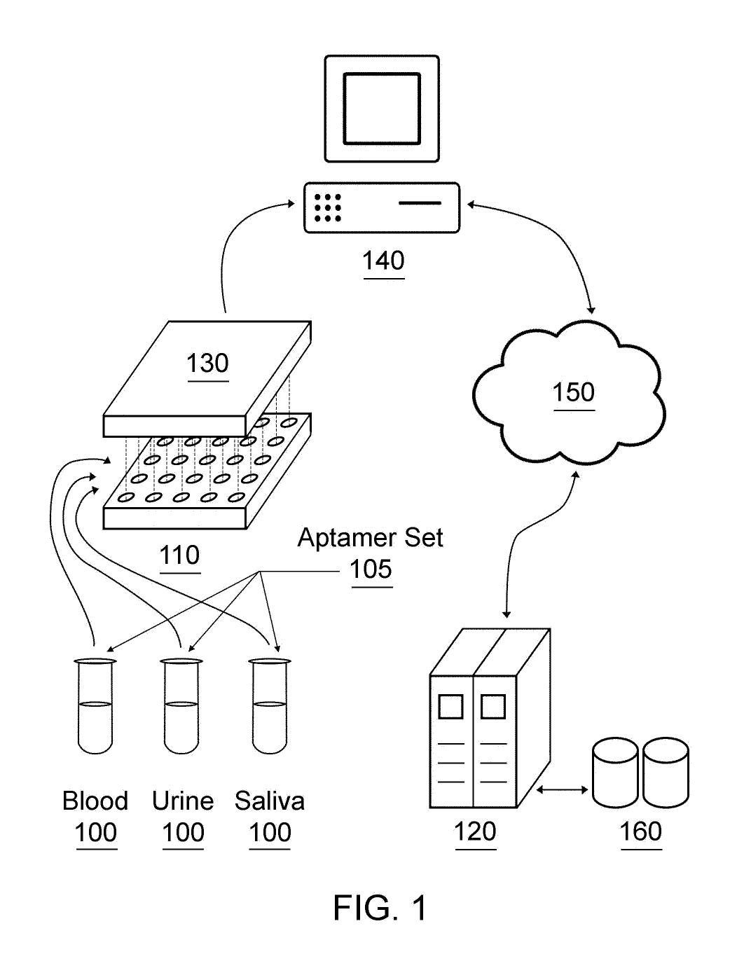

FIG. 1 illustrates an example of a system implementing the disclosed CDSS platform in order to diagnose whether a particular patient has a chronic disease. First, samples must be taken from the patient or provided to the technician performing the screening. The CDSS may utilize data from blood, urine, and saliva samples 100. Additionally, other bodily fluids could be utilized as well, and various combinations of the samples 100 could be utilized. In some examples, a blood, urine, or saliva sample 100 alone may be utilized. For instance, diagnosis of certain chronic diseases may only require one sample 100 of one type of bodily fluid that contains biomarkers that are more relevant to that particular disease. Additionally, some of the bodily fluids may have certain portions filtered, centrifuged, screen or processed before biomarker detection. For instance, serum alone could be extracted from a blood sample 100 for testing.

Next, the chosen sample(s) 100 from the patient reacted with a specific set of aptamers 105 (e.g. 5,000 different types of aptamers, or aptamers with 5,000 different nucleic acid sequences) or single stranded nucleic acids that bind to biomarkers in the samples 100. The samples are either first mixed together, or reacted separately with the aptamers. For instance, a sample of blood, urine, and saliva from a patient may be mixed and then reacted with an aptamer set. Each of the 5,000 aptamers (in this example) in this aptamer set 105 will only bind (e.g. have a high affinity and specificity) for one biomarker (one biomolecule in the sample—for example a specific protein).

In various embodiments, the aptamers in the library may have 5, 6, 7, 8, 9, 10, 11, 12, 13, 14, or 15 nucleotides, or a combination thereof. In one embodiment, the aptamer library comprises aptamers of 10 nucleotides, and wherein the 10 nucleotides are randomly synthesized; as such, some aptamer libraries can have up to 410 aptamer sequences or more if more nucleotides are utilized. In some embodiments, one may designate certain positions in the aptamer to be particular nucleotides and randomly choose nucleotides for other undesignated positions.

After the aptamer set 105 is added to the sample 100, the aptamers will bind with their target biomarkers and form complexes. The aptamers in the set 105 that do not bind to a biomarker in the sample 100 will be washed off and discarded. In some embodiments, this can be performed by first binding the sample biomarkers to a membrane, where the biomarkers for connections with the membrane. Then, the aptamer set 105 can be added, forming a membrane <=> biomarker <=> aptamer chain. The unbound and/or weakly bound aptamers can then be washed off using a buffer solution or other solution. Afterwards, the membrane can be treated with a buffer that will unbind the aptamers, and those aptamers can be separated as the bound aptamers for a particular sample 100. Accordingly, after this initial process, a pool of aptamers that bound to biomarkers in the sample is obtained.

In some examples, the sample binding aptamers can be amplified using RT-PCR or other processes and labeled with fluorescent dyes. Accordingly, a pool of florescent tagged sample binding aptamers can be obtained.

The tagged sample binding aptamers can then be applied to each will of an array 110 or other set of wells. An array 110 could be a biochip, a slide, or other solid substrate on top of which an array of spots, or spatially discrete locations for reactions as described. For instance a glass or silicon slide may include spots for several different reactions, each that tests for a different biomarker (using a different one of the aptamer sets). Accordingly, a biochip or array 110 may be any solid surface to which molecules may be attached through either covalent or non-covalent bonds. This includes, but is not limited to, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is contemplated. This includes planar surfaces, and also spherical surfaces. Preferably, these groups are then covalently attached to crosslinking agents, so that the subsequent attachment of the nucleic acid ligands and their interaction with biomarkers or sample binding aptamers will occur in solution without hindrance from the array 110 or biochip. Typical crosslinking groups include ethylene glycol oligomer, diamines, and amino acids. Any suitable technique useful for immobilizing a nucleic acid ligand to an array 110 is contemplated by this invention. In other embodiments, an array 110 could be implemented using separate wells or containers for each aptamer.

Each well of the array 110 can include a pool of immobilized identical single stranded nucleic acids (“ssNA”) with a sequence that is complementary to one of the aptamers in the set of 5,000 aptamers 105. The pool of identical ssNA that are complementary to the biomarker binding aptamers are bound to the surface of the well or tray spot.

Therefore, when the tagged aptamers are applied to each well, only tagged aptamers with sequences complementary to the immobilized ssNA will bind to that particular row and column in the array 110. Therefore, the amount of aptamers that bind to the immobilized ssNA in each spot on the array 110 is a proxy for the concentration of one particular biomarker binding aptamer. Because the sequence of each complementary ssNA acid attached to the array is known, the particular aptamer that binds to that sequence and therefore the ssNA in that spot can also be determined.

After applying the fluorescent tagged aptamers to each well, the unreacted aptamers may be washed off from each well. Accordingly, each well will contain only the fluorescent tagged aptamers that bound to immobilized ssNA's with complementary sequences.

After washing, a scanner 130 or other image detector may scan the array 110 to record image data of the array 110. The image data will include, in this example, the fluorescence intensity of each spot in the array 110. Any suitable scanner 130 may be utilized, including several of the microarray reading scanners available from Innopsys. For instance, Innopsys sells various microarray scanners that scan arrays 110 and detect fluorescence in each well (e.g. InnoScan 910, (nnoScan 710-IR, etc.) In some embodiments, these scanners may include an excitation laser, or other excitation energy source, that can be directed to the sample so that they emit detected fluorescence wavelengths. These scanners 130 may also include a suitable photodetector, for instance a CCD camera to detect the fluorescence.

The fluorescent intensity will indicate the number of aptamers that bound to each spot on the array 110. Accordingly, based on knowing the sequence of the immobilized ssNA in each well, the fluorescent intensity can be used to determine the concentration or relative concentrations of each biomarker binding aptamer in the sample 100. Accordingly, this will indicate the relative concentrations of the biomarkers that bind to each aptamer in the sample 100. Therefore, the intensities of each well are related to the concentrations of a particular biomarker in the sample 100.

In some embodiments, only relative intensities of each well may be input into a platform for screening. In other embodiments, concentrations of the aptamers may be first calculated which then may be input into a platform for screening.

In other embodiments, electrochemical aptamer sensing may be utilized instead of fluorescence based aptamer sensing. For instance, concentrations of aptamers may be determined utilized redox-labeled aptamers for electrochemical sensing of aptamers. Different electrochemically active labels (e.g. redox compounds, enzymes, or metal nanoparticles) have been employed to relay electrical signals resulting from aptamer-biomarker binding. For instance, the aptamers may be assembled onto an electrode, and differences in electrical properties may be measured before and after introduction of biomarkers. The differences in those properties (e.g. voltage, faradaic current) can be utilized to determine concentrations of particular aptamers and therefore biomarkers.

In other embodiments, luminescence, or other methods may be utilized to determine the concentration of aptamers in each well. Accordingly, the concentration determination procedure may be varied, while the other steps in the above overview may be retained or altered as disclosed herein.

After scanning of the array 110 by the scanner 130, the images and/or fluorescence intensity data may be output to a computer 140. The computer 140 may process the image data itself, or may send the image data over a network 150 to a server 120 for processing and/or storage in a database 160. In some embodiments, preliminary processing and/or concentration determination may be performed at computer 140, while further processing and diagnosis may be performed on the server 120. In some embodiments, the database 160 may store patient data for each patient, indicating the patient identification, the patient profile including health and demographic data, the patient's health history, and link that information to the output images.

Accordingly, the CDSS platform may be refined over time as patient profile data, including clinical diagnosis by methods other the disclosed CDSS platform, may be added to further train the algorithms. The CDSS platform algorithms may take the input image data output from the scanner 130 and process the data to determine the diagnosis on a local computer 140 or a server 120 or other computer located over a network 150 connected to the scanner or other local computer 140. Accordingly, the local computer may send image data, or processed data indicating the concentration/intensity of the image data pixels or some other pre-processed data set related to the intensity or image data output form the scanner 130 over a network to a remote server 120.

Accordingly, in some cases, the server 120 may then receive the image data, and process the image data to determine a diagnosis. The diagnosis may be determined by inputting the image data into a DNN based machine learning algorithm to determine whether the patient has a particular disease or not. In some embodiments, a separate DNN based machine learning algorithm may be utilized for each disease the patient is tested.

The diagnosis may then be output from the server 120 after processing the image data using the DNN based algorithm. The server 120 may then send the diagnosis to another computer 140, the same local computer 140 or other location, or store the diagnosis and run further testing. In some embodiments, all testing may be performed on a particular sample(s) from a patient before bundling the results/diagnosis and sending to a remote computer or health care institution, or directly to a doctor. In some cases, a doctor may review the results of the CDSS platform and determine a clinical diagnosis based on the results.

In some embodiments, the sensitivity and specificity of each diagnosis for a particular disease may be included with the diagnosis for evaluation by the doctor. Accordingly, once the server 120 sends the diagnosis, the data may include data representing the sensitivity and specificity for that particular disease. In other embodiments, local software for the health care provider may indicate the sensitivity and specificity that is predetermined based on prior testing.

### Selection of Aptamers

Accordingly, based on the above overview, the aptamer set 105 utilized determines the number and types of biomolecules that will be considered in the diagnosis, Therefore, the first step is to select the appropriate aptamer set 105 used for detecting the relevant biomarkers in a patient.

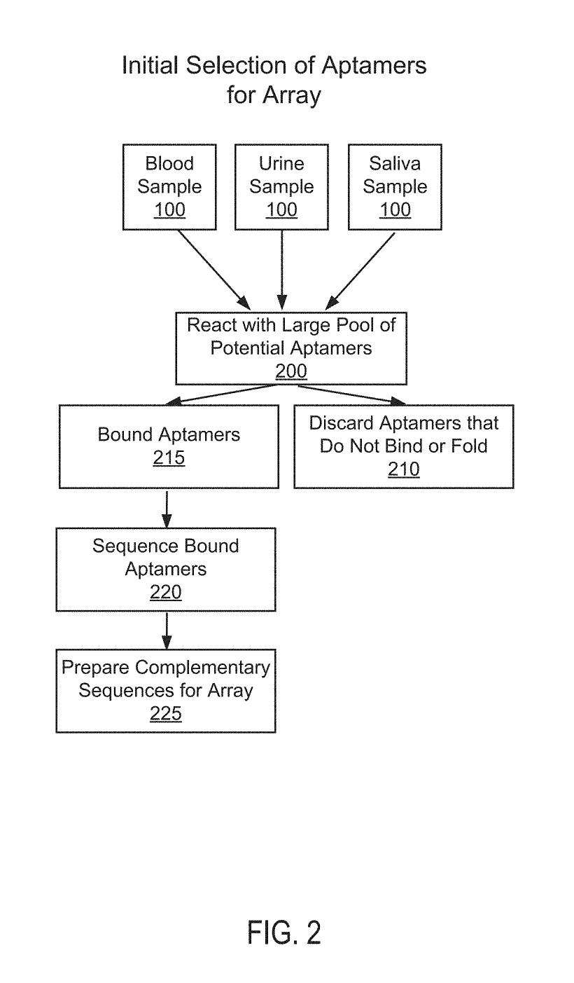

FIG. 2 illustrates an overview of that process for identifying an appropriate aptamer set 105 or sets 105 to be utilized in the disclosed system and methods. First samples 100 are taken from the patient that may include any combination of blood, urine, saliva, or other bodily fluids. Then, the sample(s) 100 are reacted with a very large pool(s) of aptamers 200 that, in some embodiments, are generated at random or are a known pool of aptamers that have affinity for biomarkers.

For instance, aptamer sequences may be randomly generated, with any redundantly similar sequences being discarded to produce an initial set of synthetic aptamers 105 to form an aptamer library 105. In this example, the aptamers may have fixed 5′ and 3′ end sequences for primers. Between the two fixed ends will be a randomized region of a target number of bases, which may have equal proportions of the four nitrogenous bases.

The initial set of aptamers 105 in the library from randomly generated sequences will be reduced based on which of the aptamers in the set 105 bind to biomarkers in various samples of blood, serum, urine, saliva, or any combination from patients. The aptamers that do not bind to any biomarkers may then be discarded 210. Accordingly, a library of aptamers that bind to biomolecules may be retained in this manner 215.

For instance, this can be performed by first applying the sample(s) 100 to a membrane (e.g. nitrocellulose blotting membrane), where the biomarkers for connections with the membrane. Then, the aptamer set 105 can be added, forming a membrane <=> biomarker <=> aptamer chain. The unbound aptamers can then be washed off 210 using a buffer solution or other solution. In some embodiments, the unbound aptamers will be washed several times to ensure that aptamers that bind only weakly to the biomarkers will also be discarded 210, so that only aptamers with high affinity will be retained 215.

Afterwards, the membrane can be treated with a buffer or other solution that will unbind the aptamers from the biomarkers, and those aptamers can be separated as the bound aptamers for a particular sample 215. Accordingly, after this initial process, a pool of aptamers that bound to biomarkers in the sample is obtained. Then, those aptamers may be amplified sequenced 220 to identify an initial set of aptamers 105 that are capable of sufficient binding affinity for biomarkers in the sample.

The source of the samples 100 that are used to screen aptamers may include the following bodily fluids: (1) blood, (2) serum, (3), saliva, (4), urine, or any combination of these fluids. Additionally, samples of each of these bodily fluids may be obtained from disease and control states, including patients of the diseases which the CDSS platform will test for, in order to select aptamers that bind to biomarkers, including those biomarkers found in chronic disease states. Accordingly, utilizing this process, aptamers that bind to relevant biomarkers can be identified.

For instance, in some embodiments, seven sets of aptamer libraries could be determined, using the different bodily fluids:

serum (blood)

urine

saliva

serum+urine

serum+saliva

urine+saliva

serum+urine+saliva

Furthermore, various combinations of these libraries may be determined for each disease state. Also, certain disease states may require their own separate library to identify for example, if the disease is particularly difficult to diagnose (and thus requires, for example, more biomarkers). For instance, after training a DNN based algorithm for a particular disease, if the specificity and/or sensitivity is not high enough, a new pool of aptamers could be generated for that particular disease.

Providing these various libraries, will allow a CDSS platform to diagnose chronic diseases based on different combinations of bodily fluid samples that are available or may not be available for a particular patient. Additionally, for certain diseases, one or a lesser combination of these three bodily fluids may only be necessary for a reduced cost.

These libraries can be used by themselves or in combination with each other, such as using the serum-specific library to analyze a serum sample, or using both serum-specific and urine-specific libraries to analyze a combined sample, or any other combination of samples.

Manufacture of the Biochip Array

Once the number of aptamers has been reduced, a microarray or biochip 110 consisting of single stranded nucleic acids (“ssNA”) with sequences that are complimentary to the identified set of aptamers 105 may be produced. In some embodiments, the ssNA will be chemically bonded to the biochip, so that they may bind to the aptamers with complementary sequences. Accordingly, the biochip will be pre-bound to ssNA, which will be capable of binding to the aptamers with the complementary sequence.

After selection of aptamers by multiple positive and negative selections (control), washing, the biochip designed is based on the complementary sequence of these aptamers. Single stranded nucleic acid (complementary sequences to aptamers) are prepared by conducting PCR on single stranded nucleic acid cloned plasmid+Fw-primer and 5′+Re-primer then the standard PCR is performed on the plasmid. Microarray/biochip materials such as aldehyde coated glass slide (CSS from BMS, Inc.) can be used to manufacture array such as pin method or biotin coated methods from a commercial company such as GenPak, Inc.

After construction, the aptamer array 110 can be used for measuring reactivity and collecting data based on the reactivity of the aptamer to the array 110. Aptamers will be reacted with different type of groups of samples (disease or control) for identifying the binding specificity to specific sample types. Again, the aptamers are mixed and incubated with specific sample types, the unbound or weakly bound aptamers are washed off, and the bound aptamers are eluted, amplified and reacted with complementary sequences on the biochip.

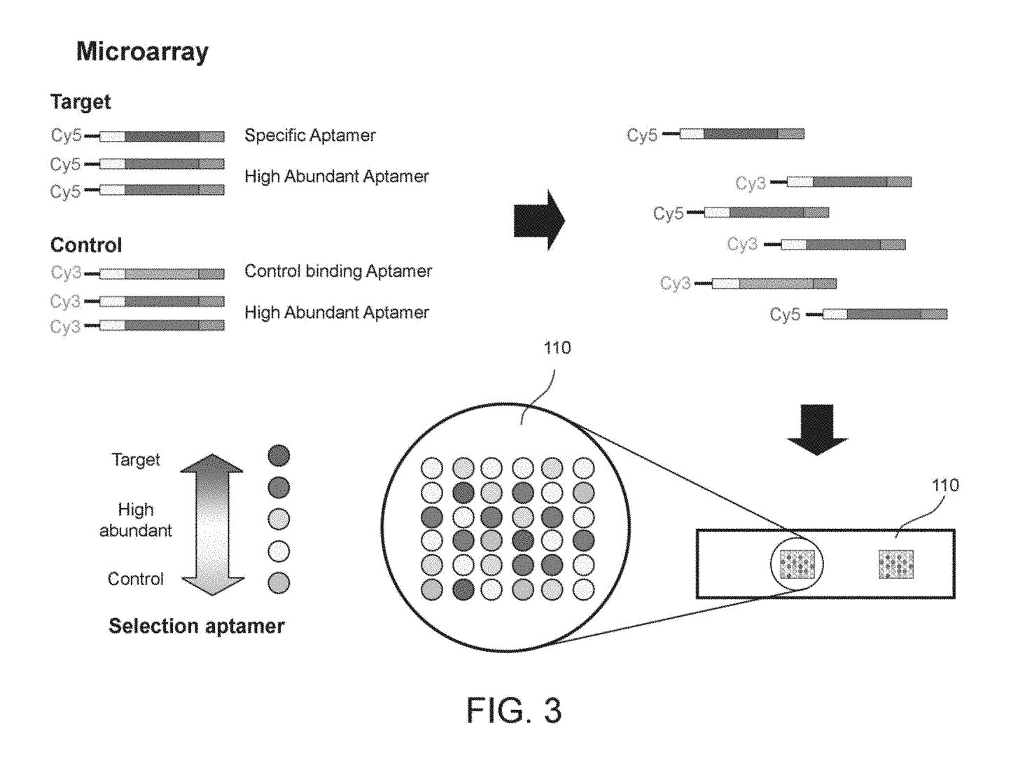

FIG. 3 illustrates an example of the aptamers and controls spotted on the array 110. As discussed, the microarray 110 is first deposited with, in rows and columns, several, hundreds, or thousands of spots, with each spot including a pool of many complementary, single stranded nucleotides (“ssNA”), all with the same sequence that uniquely binds to a specific aptamer. When the complementary ssNA are spotted on the array 110, they are immobilized or attached to the glass or other material of the array substrate, usually by some kind of chemical bond. A summary of some of these methods for fixing ssNA to the array are included in Kumar, et al. “Silanized Nucleic Acids: a General Platform for DNA immobilization.” In many embodiments, this involves coating the glass, silicon, or other substrate with a chemical substance that forms a chemical bond with the non-reactive portion of the ssNA.

Accordingly, as illustrated in FIG. 3, after fixation of the complementary ssNA, the array 110 (or biochip, microarray) is ready for utilization in testing. Accordingly, as discussed above, once a test subject's sample 100 has been hybridized with aptamers, the unbound or weakly bound aptamers washed off, and the bound aptamers freed, the bound aptamers can be amplified (e.g. RT-PCT) and labeled using a fluorescent labeled primer (e.g. Cy5 or Cy3) or other process so that they can be visualized upon hybridization.

Accordingly, the fluorescent labeled aptamers from the sample(s) spotted on the array 110 can then be allowed to hybridize with the complementary ssNAs fixed on the slide, and then the aptamers that do not bind to a complementary ssNA can be washed off. Accordingly, once spotted on the array 110 and only the hybridized aptamers remain, a UV excitation light or other excitation light can be utilized to record the intensity data of the fluorescence or other radiation of each spot on the array 110. As illustrated in FIG. 3, target and control labeled aptamers may be spotted onto the array 110, for control purposes and reference concentrations.

Because each spot on the micro array 110 has a coating of immobilized ssNA with the same sequence, and each (fluorescence tagged) aptamer bound to that immobilized ssNA will increase the fluorescent intensity in that spot. Thus, the fluorescent intensity or other concentration indicator at each spot is a proxy for the amount of biomarkers (or biomolecule) that bound to that specific sequence of aptamer. This is because the higher the concentration of the biomarker in that samples, the more aptamers that bound to the biomarker, and therefore the more tagged aptamers that will bind to the immobilized ssNAs in that spot on the array 110.

Thus, with the use of an array 110, quantification for the amount of each aptamer that bound to each sample 100 will be possible, and the selected number of aptamers 105 will be refined further to accommodate the size of the array 110 with the following method.

In some embodiments, the intensity of the fluorescence from each spot will be assayed as a measure of the amount of each aptamer, using a laser scanner or other imaging device with a corresponding frequency, for example, in the case of Cy5, 635 nm. Because each spot on the microarray 110 is associated with a specific aptamer sequence, the sequence information and the fluorescent intensity associated with that spot for each sample is recorded. After proper data preprocessing (quality control and normalization), a combination of observational and statistical criteria can be used such as average spot density threshold, fold change threshold and p-values and/or q-values based on a Statistical Variance Test, such as ANOVA to identify a set of significantly expressed aptamer targets for level of confidence detection. In some embodiments, a new array 110 will be built using oligonucleotides with complementary sequences to the selected aptamers.

In other embodiments, aptamers will be selected or further screened by a difference in expression between disease and control states using statistical criteria such as average spot density threshold, fold change threshold and p-values and/or q-values based on a Statistical Variance Test, such as ANOVA to identify a set of significantly expressed aptamer targets for level of confidence detection.

### Generating Input Data to Train the CDSS Platform

The next step in development of a CDSS platform to diagnose chronic diseases based on the concentration of biomarkers, is to “train” a DNN based machine learning algorithm with data from patients with known disease states. Thus, the algorithm can compare the concentrations of diseased and control patients to that of unknown patients to output a diagnosis.

Accordingly, after development of an array(s) 110 with appropriate aptamer (and thus biomarker) targets, the array 110 may be utilized to detect concentrations of those biomarkers in samples 100 from patients. Accordingly, if these already patients have known disease states, these concentrations may be utilized as input data to train the disclosed DNN based machine learning classifiers so that they may eventually diagnose unknown samples.

Accordingly, blood, saliva, and urine samples 100 can be collected from subjects, and then the serum, saliva, and urine will be processed separately and/or in any combination (blood, urine, saliva, blood+urine, blood+saliva, urine+saliva, blood+urine+saliva), and each combination will be analyzed systematically to generate input data to train the CDSS platform and corresponding DNN based machine learning algorithm.

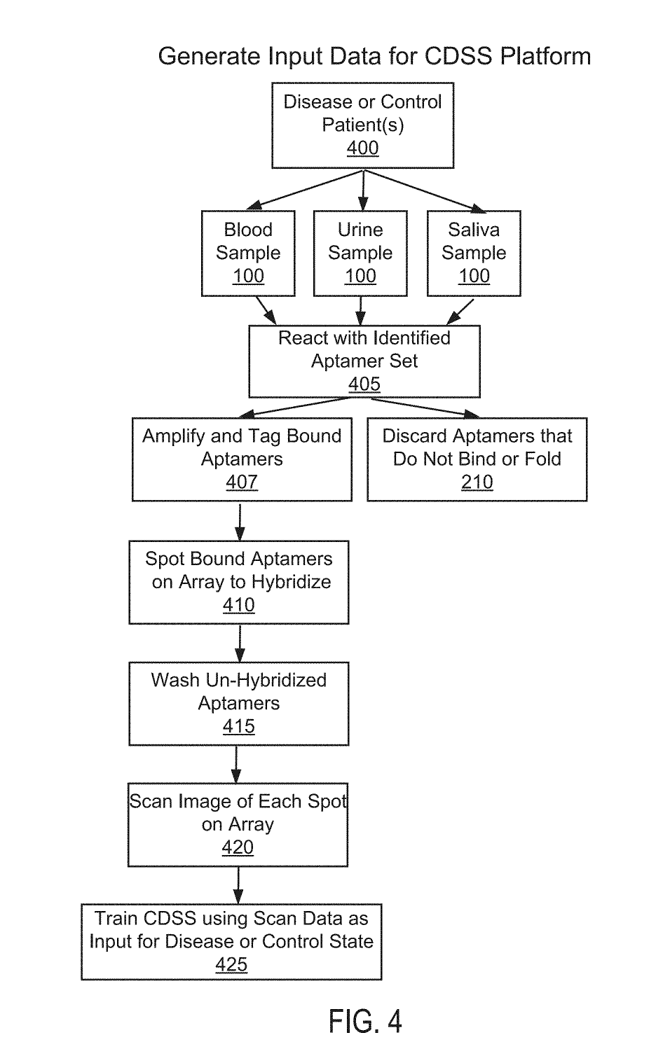

FIG. 4 provides an overview of that process. First, samples 100 of blood, urine and saliva are drawn from a subject, with a known disease (or not) state. Then, a small amount of sample 100 is dispensed on a binding platform, such as a NC (nitrocellulose) or polyvinylidene difluoride (PVDF) membrane. The membrane binds and immobilizes the sample 100 (and proteins and other biomarkers in the sample 100)

Then, the aptamer set 105 is applied to the membrane with the immobilized sample biomarkers, allowing the aptamer set 105 to hybridize with the biomarkers that are in the sample and immobilized on the membrane. Then, the membrane is washed to remove un-hybridized aptamers that do not hybridize with a biomolecule on the membrane 210.

Then, the aptamers that bound to biomolecules, are un-hybridized, and amplified using a fluorescent tagged primer to result in a set of tagged, biomarker binding aptamers. For instance, the amplified aptamers can be also labeled with indicators such as CY3 and CY5 dyes; these are cyanine dyes, with CY3 fluorescent yellow-green, and CY5 fluorescing red.

The tagged aptamers are spotted to each well on the array 410. Accordingly, in each well, the tagged aptamers that have a sequence complementary to the fixed ssNA sequence will hybridize with the ssNA. Then, after hybridization, the array 110 is washed or other process is utilized to remove unbound aptamers from the array 110.

On each spot, the aptamers that are complementary to the specific sequences of the ssNA fixed to a particular spot will hybridize and attach. Since the arrangement of the array 110 is known, a scanner 130 can be utilized to scan the array 110 in order to build an aptamer profile of that sample by sensing the intensity of the fluorescence (or other indicator) at each spot on the array. As described herein, the intensity of fluorescence in each well (e.g. spot, or spatially discrete location) will be a proxy for the concentration of the aptamer that is complementary to the known sequence ssNA of the ssNA deposited in that well. Therefore, the concentration of that aptamer will be related or have a relationship to the concentration of the biomarker to which the aptamer bound in the sample 100.

Accordingly, each spot of fluorescent intensity indicates a concentration level of a particular biomarker in the sample to which the aptamer binds. Therefore, the particular biomarker (i.e. biomolecule) that is represents may be unknown or known. In some embodiments, the biomolecule may be unknown, and the data may be input as the unknown biomolecule that binds to aptamer with X sequence.

Therefore, the concentrations may be tracked by aptamer, and not by the actual biomolecule. That allows the system to be put into place before identifying each of the relevant biomolecules, and instead just testing using the same aptamer and fixed ssNA combinations. In other embodiments, each of the biomarkers tested for may be known.

A scanner 130 will output information for each well (i.e. each aptamer containing well) in the array 110. The scanner 130 may then output intensity information by pixel, including temporal and identification information, resolution of each pixel, laser excitation wavelengths (e.g. 635, 535 nm), standard deviation and normalization methods, the type of scanner, amount of laser transmission, power of the laser, emission filters if any, coordinate values of the scan regions (e.g. in pixels). Additionally, the scanner may output information for each well, including the block, column and row number, the name, the ID, the x coordinate of the center of the well, the y coordinate of the center of the well, and the diameter of the well.

Finally, the scanner 130 may output certain intensity information. For instance, the scanner 130 may output: (1) median well intensity at wavelength #1, (2) the mean well intensity at wavelength #1, (3) the standard deviation of the well pixel intensity at wavelength #1, (4) the mean well background intensity at wavelength #1, (5) the mean well background intensity at wavelength #1, (6) the standard deviation of the well pixel background intensity at wavelength #1, (7) the percentage of well pixels with intensities more than one standard deviation above the background pixel intensity, at wavelength #1, and the percentage of feature pixels with intensities more than two standard deviations above the background pixel intensity, at wavelength #1, and the percentage of well pixels at wavelength #1 that are saturation. Additionally, these may also be output for additional wavelengths. Furthermore, comparisons between wavelength and other measures may be utilized as output information and training data for each subject sample 100 input as training data into the CDSS.

The data can then be input to the CDSS Platform as training data. This could include one of the numbers form above, various combinations, or purely the images themselves without dimensionality reduction by limiting to the above or other intensity calculations. This procedure is repeated with a large number of controls and subjects that already have a confirmed diagnosis of disease. Because the samples are from subjects with an already confirmed diagnosis, the CDSS Platform will be able to use the training data to identify statistically significant patterns and differences in aptamer-biomarker binding profiles between disease and control states.

Once the CDSS Platform has been trained, a new cohort of subjects will be used to test how well it can identify and sort samples that it has previously never seen before into disease or control categories. If the sensitivity and specificity is not sufficiently high enough, training data from more subjects will be inputted until it reaches the desired accuracy and precision of predicting disease.

### Clinical Deployment

Once the CDSS Platform is sufficiently trained, it can be deployed in a clinical setting to assist physicians in screening and diagnosing patients. The process is identical to the training of the CDSS Platform as described before.

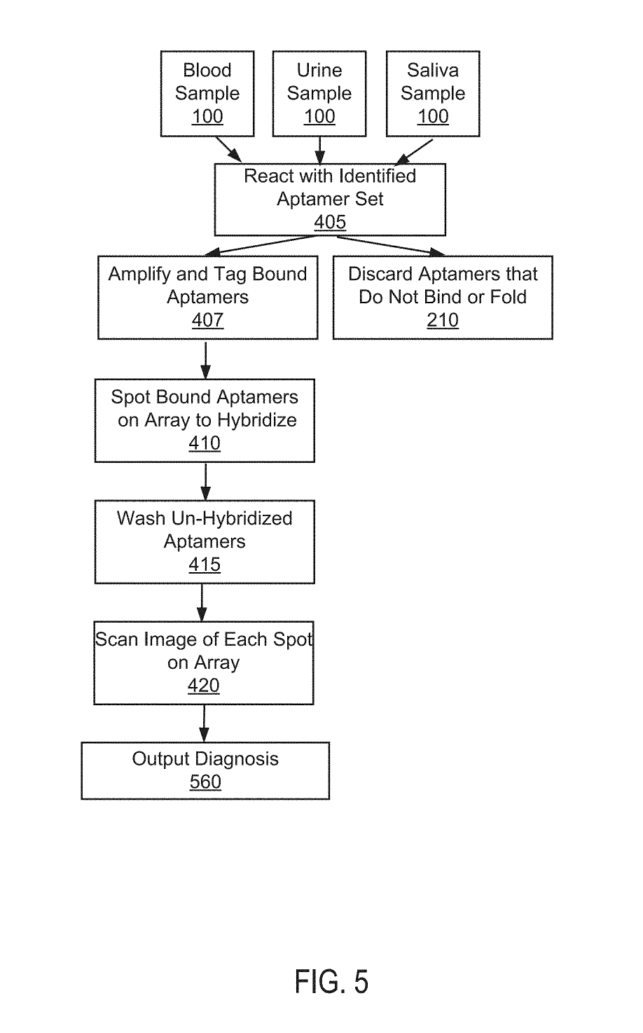

FIG. 5 illustrates an overview of that process. FIG. 5 is essentially the same process is FIG. 4 except that the disease state of the patient from which the technician drew samples 100 is unknown, and is not used as training data. Rather, the samples are input into the system to output a diagnosis of disease or no-disease 560.

Accordingly, blood serum, saliva, and/or urine samples 100 will be collected from the patient, and then the samples 100 will be processed separately or combined into a single liquid sample of any combination thereof. The groups of samples be dispensed on a binding platform such as a NC (nitrocellulose) or polyvinylidene difluoride (PVDF) membrane and reacted with the identified aptamer set 506. Then, the membrane will be washed, and bound aptamers will be extracted 407, amplified, labeled with fluorescent dyes 407. Then, the dyed aptamers can be visualized on the biochip array (410-420).

The CDSS Platform will read the fluorescent microarray image and compare the sample to its database of training data and evaluate whether the pattern of aptamer binding is more similar to a disease state or a control state and output the diagnosis 560. The CDSS Platform will then inform the physician of its analysis.

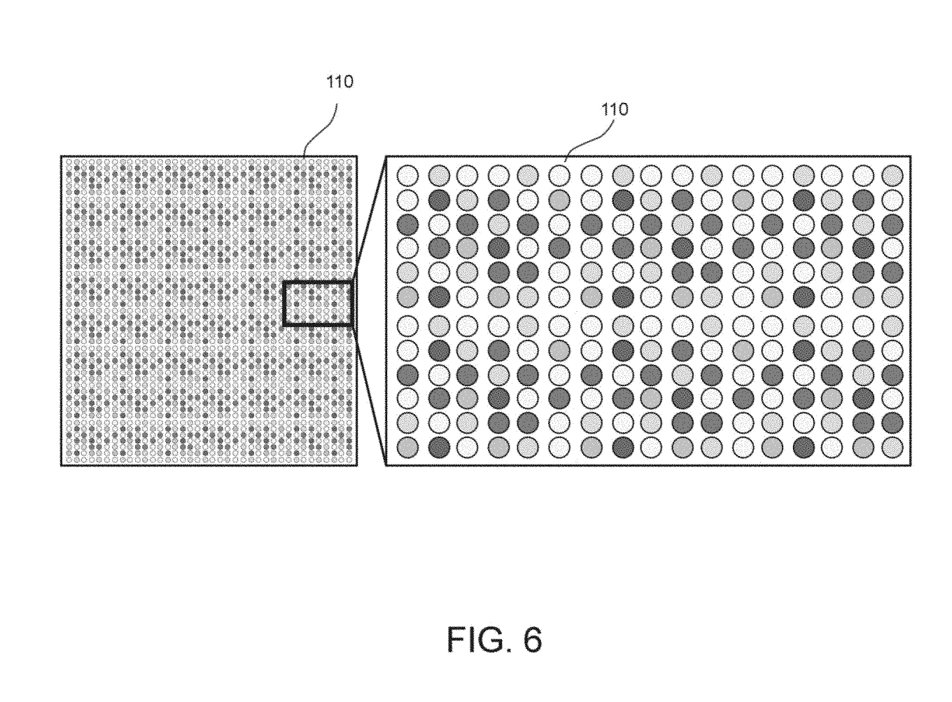

FIG. 6 illustrates an example of an array 110 and the fluorescent spots. Each of the spots (wells, dots) has varying intensities of fluorescent wavelengths.

Deep Neural Network for CDSS Platform

Accordingly, disclosed below are the algorithms for analyzing the image data output from the scanner 130, comparing it to training data, and outputting a diagnosis 560.

The CDSS Platform for the screening and diagnosis of chronic disease disclosed herein utilizes machine learning algorithms for this process that function like the neural networks in the brain. The human brain usually stores memory in 10-15 layers and in a distributed fashion—this is how it learns and solves problems. Similarly, the machine learning classifiers utilized by the disclosed systems incorporate a number of hidden layers, and like a human, have the ability to learn and improve its performance. For example, the classifiers improve as more test samples 100 are entered and are verified by clinical diagnosis.

Current machine learning algorithms utilized for diagnosis of diseases utilize a limited number of biomarkers (e.g. 30) or utilize tissue extracted from the diseased area (e.g. tumor tissue biopsied) in order to diagnose chronic disease like cancer. However, when using a larger number of biomarkers (e.g. 50, 100, 1,000, 5,000 or more) the machine learning algorithms are not sophisticated enough to model the complex processes of the biomarkers and minute concentration differences. Additionally, human physiology is so different between individual and dynamic in each individual, it is extraordinary difficult to implement a satisfactory machine learning algorithm that results in sufficiently high (or superior) sensitivity and specificity. Additionally, many diagnostic platforms utilizing machine learning algorithms rely on subjective inputs, such as analyses of tissue histology, or symptoms. Accordingly, so far researches have not developed platforms sufficiently robust to diagnose chronic diseases utilizing biomarkers that are relatively large in number (or developed systems for identifying an optimal set of biomarkers as disclosed above).

However, there is a relatively new type of machine learning algorithm that is capable of modeling very complex relationships that have a lot of variation that are called deep neural networks. Deep neural networks have developed recently to tackle the problems of speech recognition. However, they have not yet been applied for the diagnosis of chronic diseases. This is because it has not been discovered: (1) how to isolate a large number of optimal set of biomarkers for reliable detection, (2) current solutions all rely on biomarkers that have a documented association between the disease (biomarker bimolecular pathways) and thus large number of biomarkers are not utilized, (3) deep neural networks have not yet been tried practically in the diagnostic field to ascertain the potential for success in a quite unpredictable field. Accordingly, the inventors have discovered that applying the DNN to a large set of the the most relevant biomarkers allows for highly sensitivity and specificity in diagnosing chronic diseases. This is particularly revolutionary for chronic diseases, which are largely involved with and trigger uneven balances in many of the body's natural processes and natural biomolecules (i.e. biomarkers).

In the IT industry fields, various architectures of DNN have been proposed to tackle the problems associated with algorithms such as ANN by many researchers during the last few decades. These types of DNN are CNN (Convolutional Neural Network), RBM (Restricted Boltzmann Machine), LSTM (Long Short Term Memory) etc. They are all based on the theory of ANN. They demonstrate a better performance by overcoming the back-propagation error diminishing problem associated with ANN.

Back-propagation error is defined as the error between target and actual output of ANN. A traditional ANN drawback is that it gets stuck in local minima and produces weights that can't be converted into meaningful output data. Accordingly, DNN can be used to train each layer of the CDSS platform. This method solves the problem preventing the system getting stuck in a local minima which allows it to generate meaningful weight data.

A DNN algorithm with human serum, urine and saliva, or any combination of these samples/biomarkers for input data has never been applied before for the purpose of disease screening and diagnostics. This is the first time DNN has been used for this purpose.

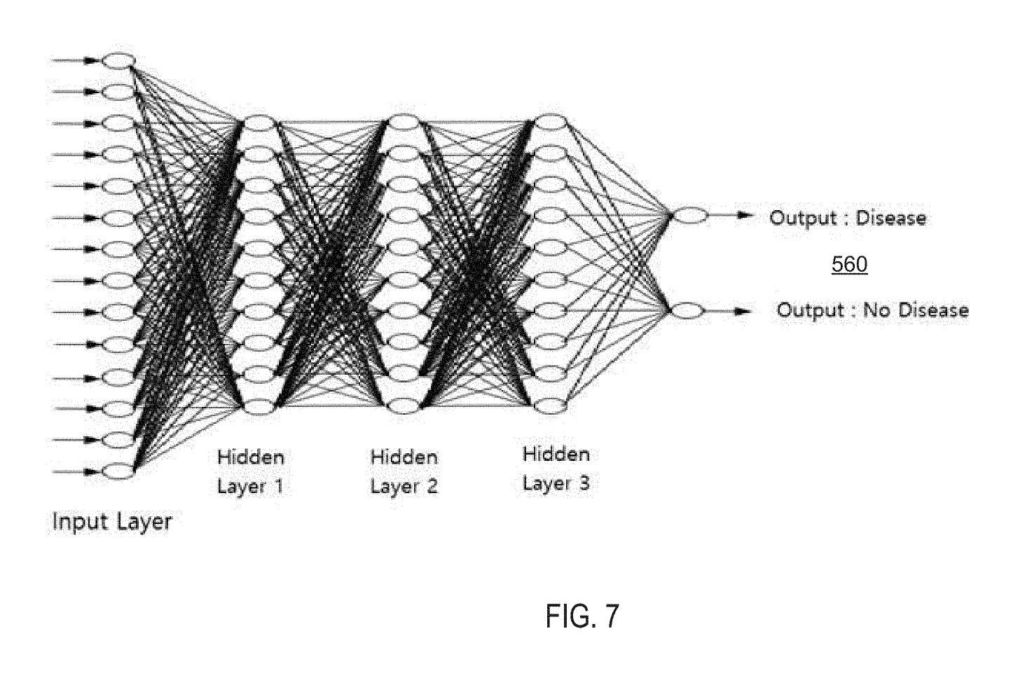

The inventors are using DNN as the backbone of the CDSS platform utilizing a number of hidden layers and each layer is modeled using algorithms such as RBM, CNN, LSTM, etc. DNN is capable of learning the probability distribution over all of the sets of inputs. For example, FIG. 7 shows the structure of an RBM based DNN used to diagnose whether a patient has a disease.

As illustrated in FIG. 7, input data is generated from reaction between the selected aptamers and the bound corresponding biomarkers in the human samples from blood, urine and saliva, or any combination of these human samples. The output represents the decision made by the CDSS Platform. The possible outputs are either (1) disease or (2) no disease and are based on the input data.

Each layer is interconnected with weights, Wij(l) (W=weight, l=total number of layers, i=total number of nodes represent a higher level of representation of selected aptamers that bind to the corresponding biomarkers known in the present layer, j=total number of nodes represent lower level of representation of selected aptamers that bound to the corresponding biomarkers known from the previous layer from layer to layer).

This process is for optimizing the weight values associated with the hidden layers. The weight associated with Input layer 1 is optimized by the output of hidden layer 2. The weight associated with hidden layer 2 is optimized by the output of hidden layer 3. Then the process is started back at hidden layer 1 and proceeds to hidden layer 3 again. Once the number of selected iterations is met the process finalizes by going to the final output layer.

Training of DNN results in the optimum weight values that will most accurately represent the highly nonlinear functional relationship between input (e.g. image data output from the scanner 130) and output data (e.g. disease or no disease). The final relationship between various data such as physiological changes (input) in patient to the decision of disease or no disease of highly complicated chronic diseases (output) can then be determined using the CDSS platform.

### Generating Input Data for the CDSS Platform

As described above in FIG. 4, input data will be prepared from samples 100 of blood, urine, and saliva, or any combinations of these human samples taken from many human subjects for each target disease. These samples 100 will be collected from each human subject. They will then be reacted with the selected aptamers that have firmly attached to the corresponding biomarkers to generate the reaction data. The reaction data derived from each human subject and will be used as input data to train the CDSS platform 425. As described above, reaction data will be related to the concentration of the biomarkers in the sample 100, and may include fluorescent images, intensity, electrochemical voltages, and other indications of biomarker concentration based on the assay utilized.

The CDSS Platform can have a number of hidden layers. For instance as illustrated in FIG. 7, a CDSS Platform may utilize three hidden layers. The raw input training data (input Layer) has a slightly higher level of representation than the raw input data. The next layers are composed of hidden layer 1, hidden layer 2, and hidden layer 3, which designate successively higher levels of representations. The output layer 560 is the final layer and provide an output of disease or no disease 560 and represent the highest level of representation.

The purpose of supervised training and unsupervised training allows the CDSS platform able to learn the highly nonlinear functional relationship between input and output data. In this application, input data represents the reaction data of selected aptamers that have firmly attached to the corresponding biomarkers found in human samples (blood, urine and saliva, or any combination of these human samples) and the output data represents the decision of disease or no disease based on each input data 560.

Training of the CDSS Platform can be achieved by using CNN based DNN, RBM based DNN, or a Combination of CNN and RBM based DNN. The combination of CNN and RBM based DNN can be achieved by training each of them separately, and then the output results can be combined. Each training method can be classified to produce optimum output result. The outputs of both CNN and RBM based DNN can be merged to produce the final classification (disease or no disease) result. For instance, an RBM based algorithm may incorporate the output of the scanner 130 as the dimensionality reduced intensity data (e.g. the values discussed above). Contrarily, CNN based DNN platforms can utilize the raw image data for each pixel that includes richer information about the intensities to extract subtler differences and more dimensions of intensity differences between the different spots on the array 110.

Accordingly, both may be combined for an even more accurate and robust diagnosis. The reduced dimensionality of the RBM based DNN may provide the robustness of relying on key intensity metrics (e.g. mean & median intensity per well/spot), and the CNN based DNN may pick up finer discrepancies between concentrations of key biomarkers (through concentrations of aptamers) to increase the sensitivity or specificity further perhaps. Below, methods for training both a (1) RBM based DNN and (2) a CNN based DNN are disclosed.

### Training the CDSS Platform Using an RBM Based DNN

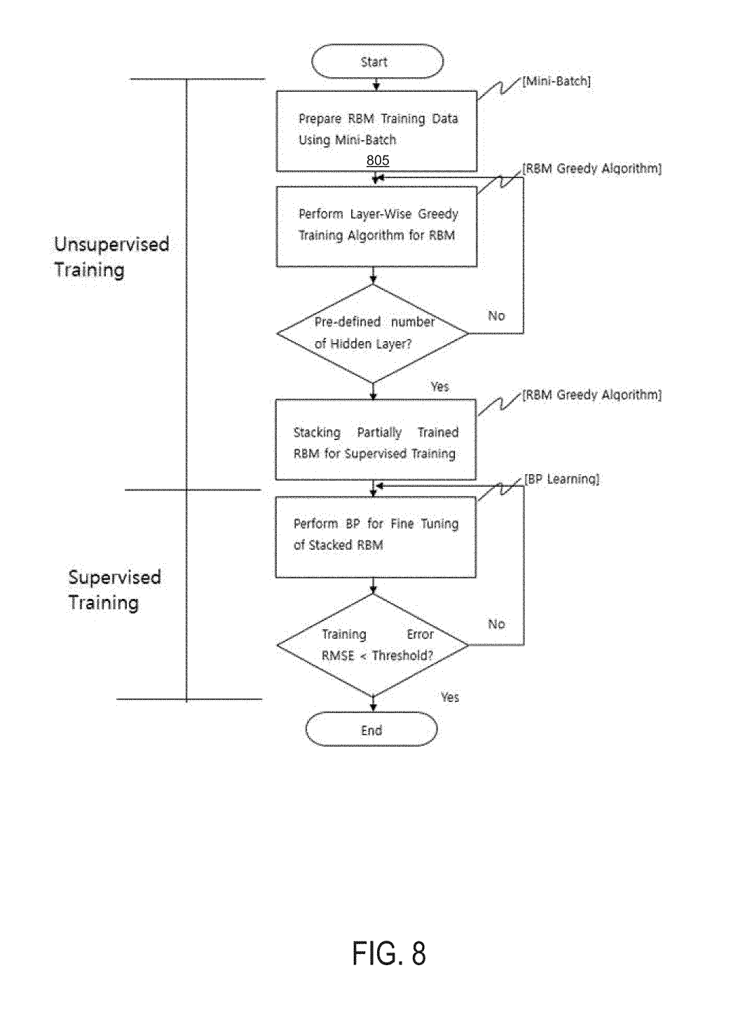

FIG. 8 depicts RBM based DNN, and each training step that consist of both unsupervised training steps and a supervised training step. From a high level, the unsupervised training is used to train the CDSS platform and the supervised training is used to fine tune the CDSS platform.

During the training stage of the CDSS platform, instead of updating the weight after estimating the gradient descent on a single training data which consists of the total number of aptamers that firmly attached to the correlated biomarkers selected from one human samples, it is better to group the training set into small mini-batches 805 and update the weight with the average gradient descent of all the mini-batch 805 training data sets.

Using the average mini-batch gradient descent may identify the best local minima that represents the sub-optimum weight solution compared with using a single data gradient descent. Each mini-batch 805 can be composed of a portion of an entire training data set. For example, in the case of the mini-batch 805 having 100 input data out of 200,000 total number of training data, then about 2,000 mini-batch sets are used for training the CDSS platform for the sub-optimum weight solution. Once the mini-batch process 805 has been completed, then each of the hidden layers should be trained using the unsupervised RBM algorithm.

For example, one mini-batch can be composed of either 100 blood serum, 100 urines samples, 100 saliva samples, or 100 samples of any combination of these human samples.

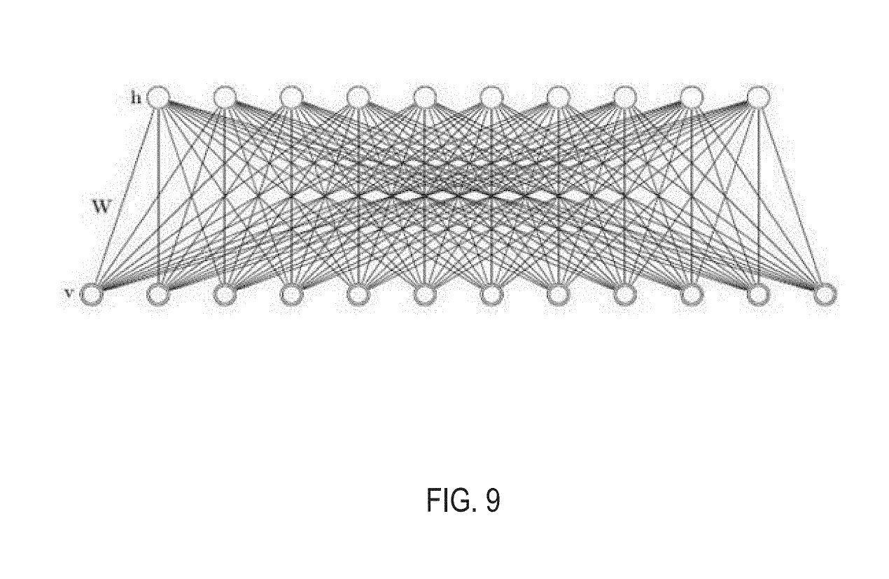

FIG. 9 depicts the structure of the RBM algorithm used to train each hidden layer. The purpose of these processes are to diminish overall training errors by adjusting the weight values to better represent the nonlinear functional relationship between input and output data. In FIG. 9, v represents all inputs, w represents weight parameter that can be assigned with random numbers, and h represents the RBM outputs for all hidden layers:

h=F(v;w)

So here vi=v1, v2, v3, v4 . . . v5000 is Ai (iterations).

Assume that there is 5,000 aptamers that firmly attach to the correlated biomarkers. Each aptamer is designated as Xi (i=1, 2, 3, 4, . . . 5,000). Then each reaction value of the aptamer with the samples from first human subject are designated as vi (i=1, 2, 3, 4, . . . 5,000). These 5,000 sets of reaction data which are v1 for the first human sample will be the input data 1. The reaction data from the second human sample will be the input data 2 (v2). If samples from 200,000 human subjects are collected, then we will have 2,000 mini-batch sets to sub-train the CDSS Platform in the case that each mini-batch data consists of 100 input data points.

Once the mini-Batch data preparation process has been completed, then each of the input data of the mini-batch will be fed into each hidden layer to be trained using an unsupervised RBM algorithm as illustrated in FIG. 8.

In the Layer-Wise Greedy Training Algorithm, for each subsequent level of hidden layers the lowest hidden layer becomes the v (input data) of the RBM and the highest hidden layer becomes the h (output data) of the RBM. The RBM is trained using mini-batch 805 training data sets for a predefined number of iterations. The process is repeated with the entire training steps described in FIG. 8. The entire training processes is then repeated for a satisfactory number of iterations.

The RBM training paradigm is an unsupervised learning process and the main purpose of this step is not to make the final decision of disease or no disease but rather to calculate the learning error. For the training of one-layer RBM, contrastive divergence CD(1) approximation is used with a single training data. Contrastive divergence is the techniques that can be used to optimize the weight value Wij.

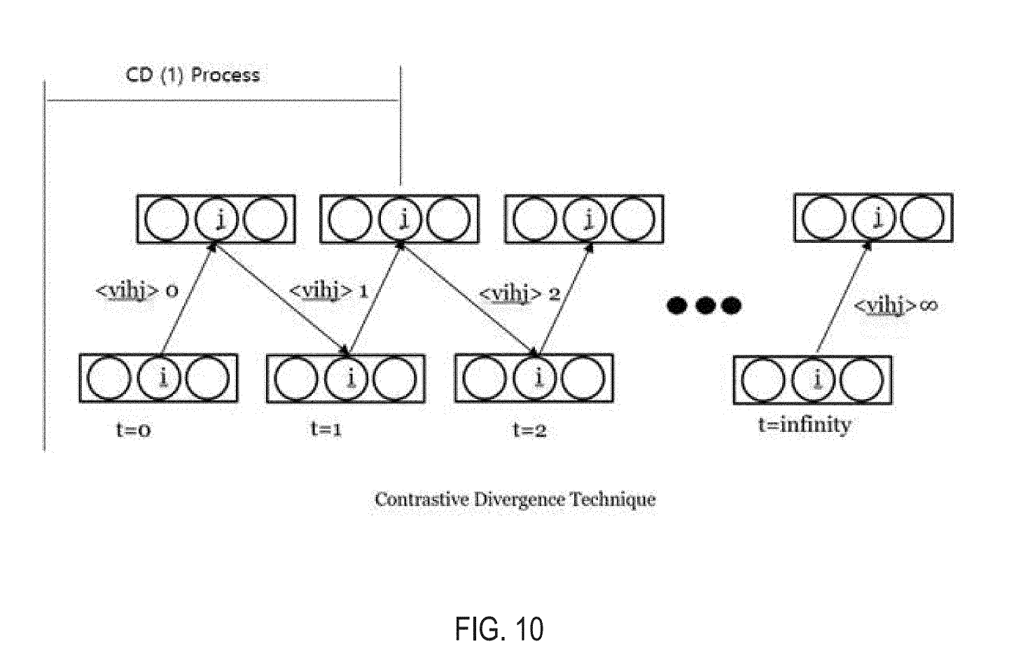

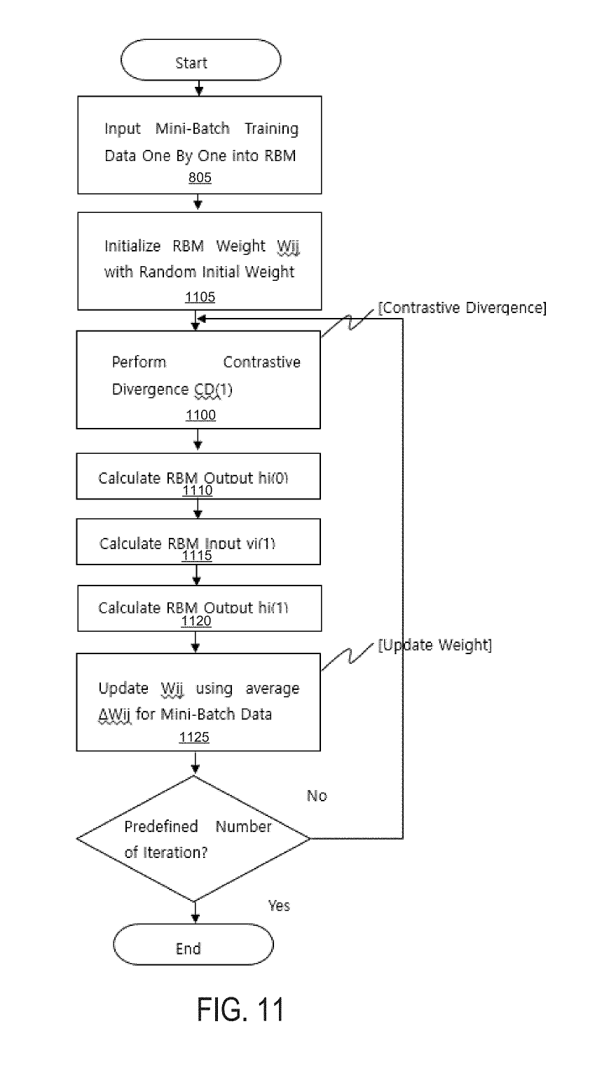

FIGS. 10 and 11 illustrate diagrams showing the contrast divergence technique for the training of a single layer of an RBM. The Contrastive Divergence CD(1) technique is used to approximate the CD(∞). To calculate the exact gradient for a single training data the CD(∞) reconstructed data is needed. It is impossible to get CD(∞) data; therefore, it is approximated with CD(1) reconstructed data 1100. First, the input vi(0) is used to calculate the output hj(0) 1110. Second, hj(0) is used to calculate the vi(1) 1115. Then, vi(1) Q used to calculate the hj(1) 1120. This alternating calculation is called the contrastive divergence technique. By this method CD(1) is used to approximate CD(∞).

The contrastive divergence (CD) algorithm which was developed by Hinton is the most often used algorithm to train the RBM. This algorithm performs Gibbs sampling and utilizes the gradient descent algorithm. It is used to train feed forward artificial neural networks using back propagation to update weight values.

The training procedures of one hidden layer of the revolutionary CDSS Platform are depicted in FIG. 11. The basic, single step contrastive divergence (CD-1) 1100 procedure for a single sample can be summarized as follows:

1. Initialize the visible units to a training vector 1105.

2. Update the hidden units in parallel given the visible units.

$$P ( hj = 1 \vert V ) = σ ( bj + {\sum }_{i}^{•} viwij )$$

σ represent the sigmoid function and bj is the bias of hj

3. Update the visible units in parallel given the hidden units.

$$P ( vj = 1 \vert H ) = σ ( ai + {\sum }_{i}^{•} hjwij )$$

σ represent the sigmoid function and ai is the bias of vi

This is called the “reconstruction” step.

4. Re-update the hidden units in parallel given the reconstructed visible units using the same equation as in step 2.

5. Perform the weight update 1125.

### Stacking RBM

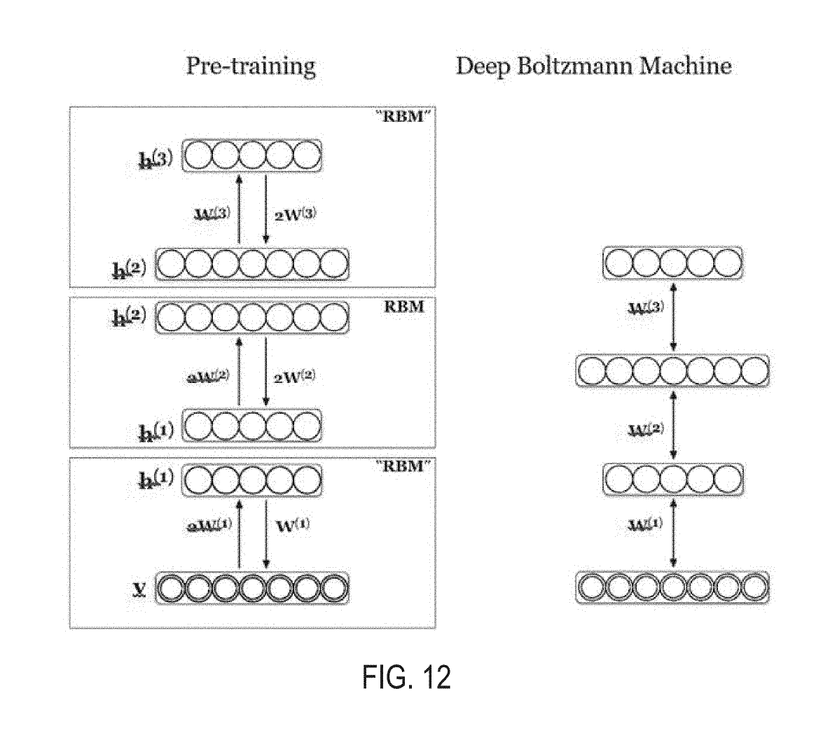

FIG. 12 illustrates a training procedure for a stacked RBM. The layer-wise greedy trained RBM's are stacked to form a deep architecture for further supervised training using a Back Propagation (BP) algorithm. After an RBM is trained, another RBM is “stacked” on top of it to create a multilayer model. Every time an additional RBM is stacked, the input visible layer is initialized to a training vector and values for the units in the already-trained RBM. New layers are assigned using the current weights and biases. The final layer of the already-trained layers is used as input to the new RBM. The new RBM is then trained with the procedure above, and then this whole process can be repeated until some desired stopping criterion is met.

### Perform Back Propagation

Stacked RBM's are further optimized using the BP (Back Propagation) algorithm that is the most widely used training algorithm for ANN. Single or Stacked RBM's can be treated as conventional feed-forward neural networks. They can model the highly complex nonlinear functions from the input layer to output layer and include many hidden layers. The top layer of stacked RBM is augmented by an output layer, where units in the new added layer represent the labels of corresponding observed training data. Then, a standard neural network for classification is further optimized by standard supervised learning algorithms, such as the BP algorithm.

The purpose of finding the optimum weights is to minimize the error between target output and actual output (Error=Target Output−Actual Output). For the training of the DNN, the training, input data (Vi, Yi) and corresponding target data Yi (Disease or No Disease) should be prepared in advance for supervised training with our RBM based DNN. When training input data Vi is fed into the DNN it is multiplied with the calculated weight (W) and results in the output (Oi). Oi=Vi×W1 (w of hiddenlayer1)×W2 (w of hiddenlayer2)×W3 (w of hiddenlayer3). The ERROR=Yi−Oi.

The purpose of the training is for finding the corresponding weights that minimize the error for every input training Vi data. The most widely used technique is called the Stochastic Gradient Descent algorithm. It is capable of searching a very complex weight space that can exist. When the training function is not simple and cannot be solved easily the Gradient Descent algorithm will change the weight in the amount of the gradient based on the error. W β W+Δ(ERROR)/Δ(W). Weight space is a very large exponential space. The actual weight space is more than the number of weights dimensional space versus the error.

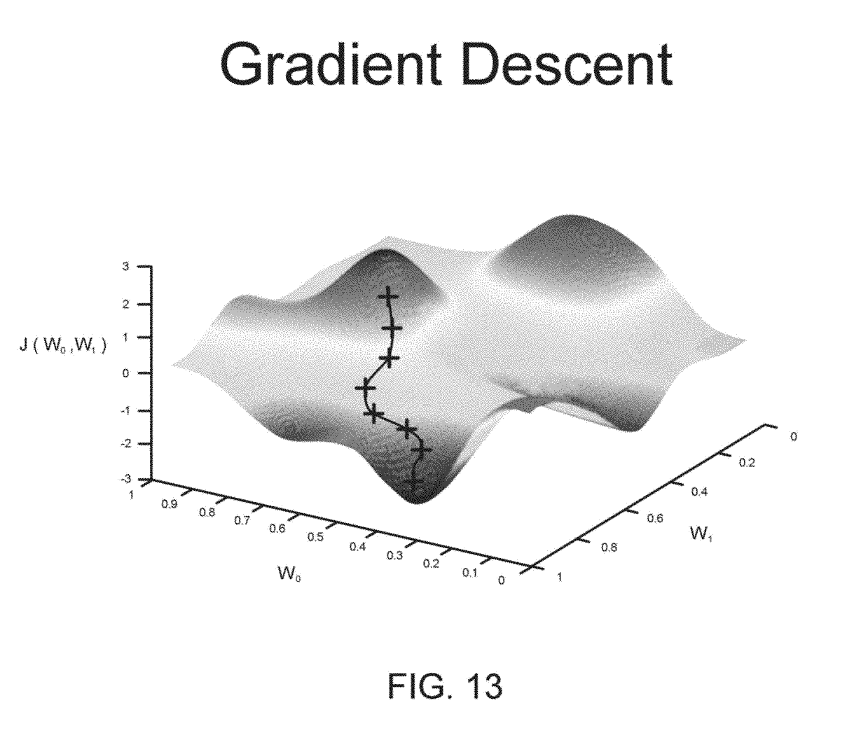

FIG. 13 depicts a 3D graphical model depicts W1 vs W2 vs qERROR. When training, the computer traverses the error space by adjusting the W1 and W2 in the amount of the gradient descent which is equal to the derivative of the error function divided by the derivative of the weight function. Gradient Descent=(Δ(ERROR)/Δ(W)).

Accordingly, FIG. 13 shows the error space versus weight (W1, W2). Optimum weight is the W1 and W2 that minimize the error. That point is represented by the darkest bottom area in the figure. The optimized error is the lowest valley or local minima. Where the Gradient Descent is equal to 0 that point is called the local minima. Actual weight space is exponentially high and is impossible to draw.

Training the CDSS Platform Using CNN based DNN

As a class of deep learning models, CNNs are known as the rst truly successful deep neural networks architecture and are specially designed for two dimensional data. We can adopt the advantages of CNNs for an aptamer profile image classi cation (disease or no disease) task and a CNN architecture is also proposed for this aptamer profile image classification task.

Convolutional Neural Networks (CNNs) are known as the rst successful deep architecture that retain the characteristics of traditional neural networks. As with the generic deep learning algorithm, the abstract level increases from layers to layers.

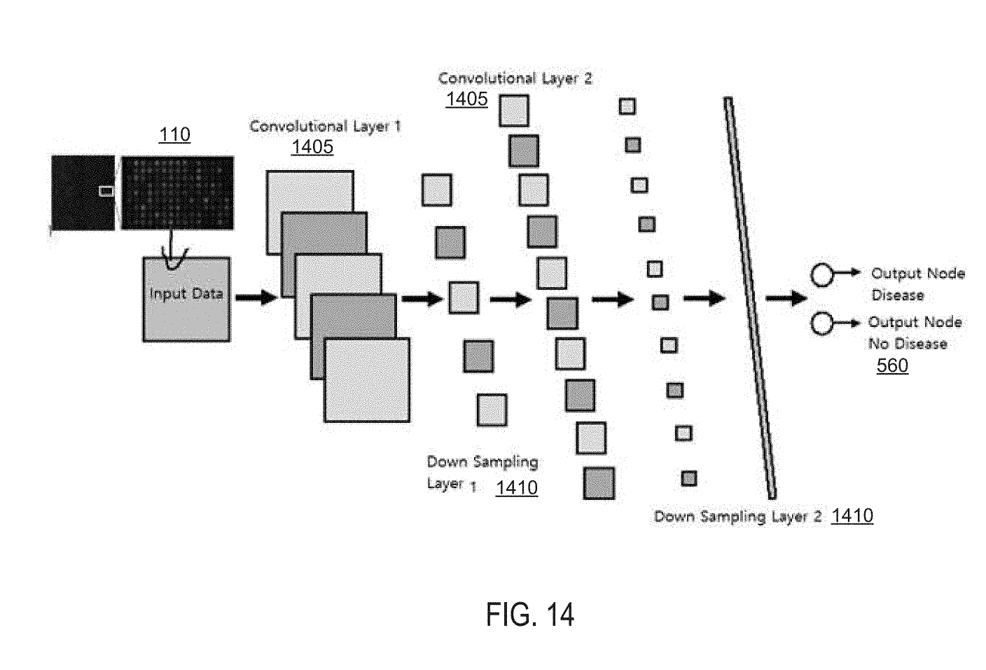

FIG. 14 illustrates an example of a CNN based DNN algorithm. Designing CNNs consists of two types of hidden layers: convolution 1405 and subsampling 1410 layers. The key idea of CNNs is that it minimizes the parameters needed to learn by pooling the lter responses over the convolution and subsampling layers to obtain a higher level abstract features.

In the CNN based DNN algorithm for the CDSS platform, mij is the function of output. The output convoluted image value from the scanner 130 from the array 110 is represented by the letter m. Each row in the layers is represented by i. Each column in the layers are represented by j.

The input image used to train the CDSS Platform using CNN based DNN is an image of the reaction data on a biochip or microarray 110 developed by the inventors. It represents the selected aptamer profiles data.

The convolution layers 1405 perform convolutions over feature maps in previous layers. The j feature map in the i layer, denoted as mij, is convoluted with some small kernels k as follows:

mij=tan h(Σn□kijn+bij)

where tan h is the hyperbolic tangent function and bij is a trainable bias for each map while kijn is the trainable kernel for mij corresponding to each map m(i−1)n.

The sub-sampling layers compute the spatial average of a small region (n×n pixels), multiply it by a weight wij, then add a trainable bias and pass through the tan h function as follows.

mij=tan h(wijΣm(i−1)jn×n+bij)

The last layer of a CNN is usually fully connected with the previous layer. It can be considered as a nonlinear classi er of features obtained by repeating the convolution and subsampling process. One of the advantages of CNNs is that we can use it in various structure design for each particular data set.

Merging Outputs from RBM and CNN Based DNN

The combination of CNN and RBM based DNN can be achieved by training each of them separately, and then the output results 560 can be combined. Each training method can be classified to produce optimum output result. The outputs of both CNN and RBM based DNN will be merged to produce the final classification (disease or no disease) result 560.

DNNs can be trained separately with different training input generated from biomarker human samples of Blood, Urine, Salive, Blood+Urine, Blood+Saliva, Urine+Saliva, Blood+Urine+Saliva for each of human samples combination. For each of human samples combination, RBM and CNN is trained separately to produce sub-optimal testing outputs. The output of RBM and CNN for each of human samples combined is integrated to produce a final optimal output. For each of classifier Ci, 1<=i<=M, the performance table Qi is used to produce final optimal output.

Qi=[q11,q12, . . . q1M q21,q22, . . . q2M . . . qM1,qM2 . . . qMM]

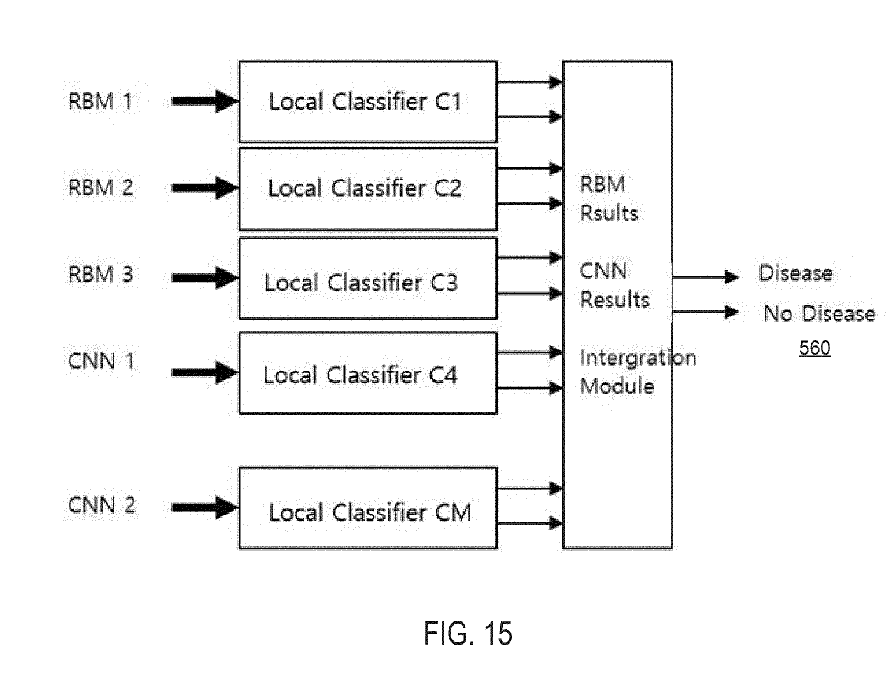

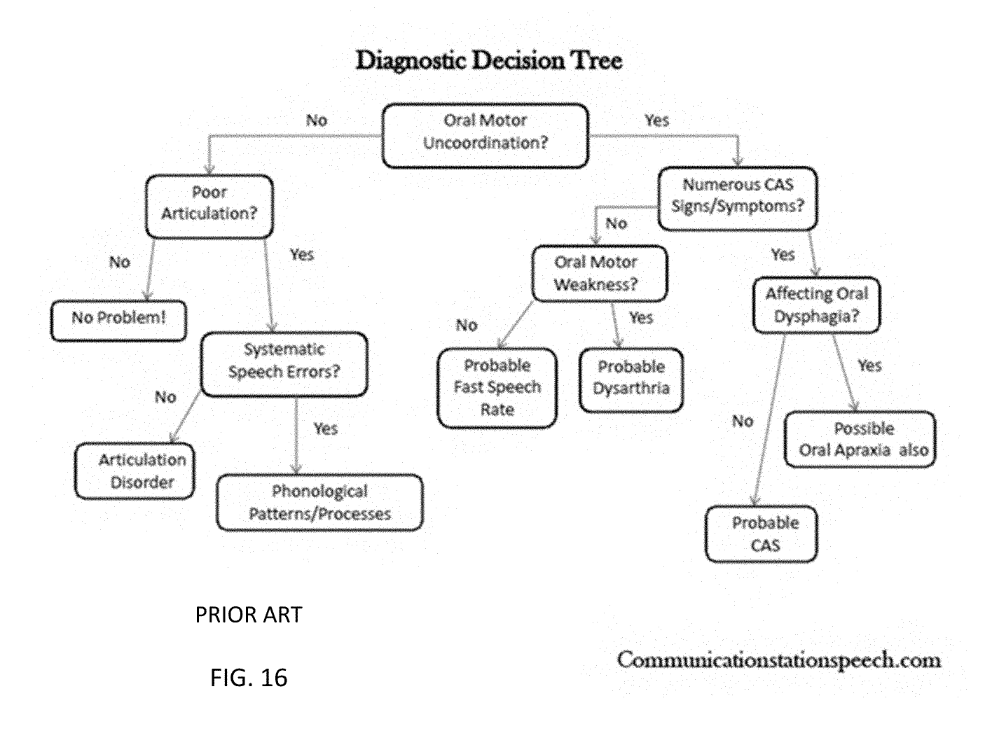

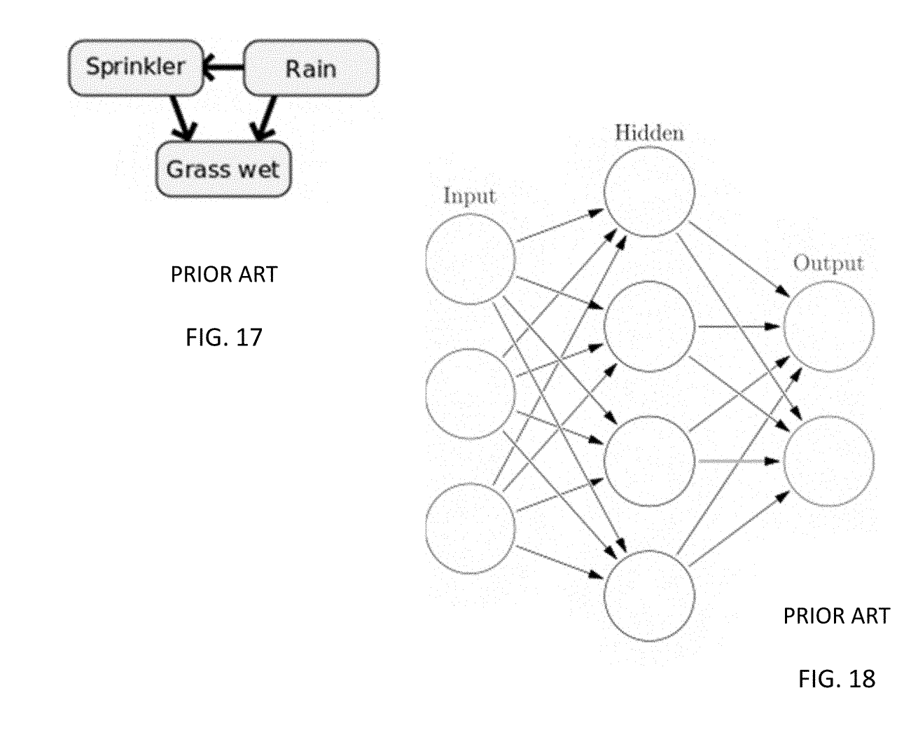

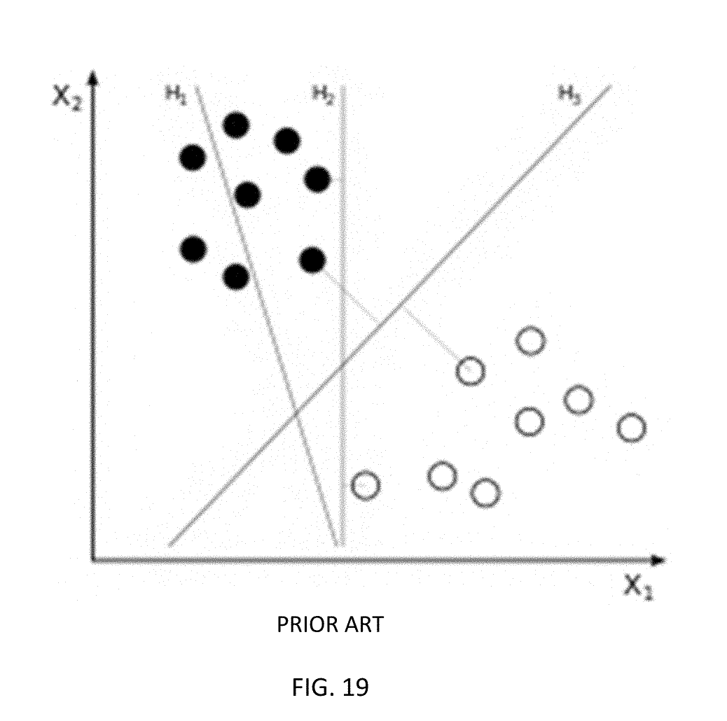

M is the number of separately trained DNN. The qij represents the performance of local classifier Ci that i-th input data is classified as j-th class. FIG. 15 depicts one example of the simplified structures of RBM based DNN and CNN based DNN combined module. The integration module 1500 may be another layer of machine learning algorithm as described herein to determine the final output of disease or no disease 560.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### REFERENCES

1) Wyatt J, Spiegelhalter D. Decision Support Systems. Open Clinical. OpenClinical. 1991. August 2015.
2) Won Y., Song H., Kang T. W., Kim J., Han B., Lee S. Pattern analysis of serum proteome distinguishes renal cell carcinoma from other urologic diseases and healthy persons. Proteomics, 2003. 3(12), 2310-2316.
3) Lynn B. Gerald, Shenghui Tang, Frank Bruce, David Redden, Michael E. Kimerling, Nancy Brook, Nancy Dunlap, and William C. Bailey. A Decision Tree for Tuberculosis Contact Investigation. American Journal of Respiratory and Critical Care Medicine. 166(8). 1122-1127.
4) Xia Jiang, Diyang Xue, Adam Brufsky, Seema Khan, and Richard Neapolitan. A New Method for Predicting Patient Survivorship Using Efficient Bayesian Network Learning. Cancer Inform. 2014; 13: 47-57. Published online 2014 Feb. 13.
5) Das, R., I. Turkoglu, and A. Sengur. Effective diagnosis of heart disease through neural networks ensembles. Expert Systems with Applications, Elsevier, 2009. 36, 7675-7680.
6) Srinivas, K., B. K. Rani, and A. Govrdhan. Applications of Data Mining Techniques in Healthcare and Prediction of Heart Attacks. International Journal on Computer Science and Engineering (IJCSE), 2010. 2(02), 250-255.
7) Sitar-Taut, V. A., Zdrenghea, D. POP, D. A., Sitar-Taut, D. A. Using machine learning algorithms in cardiovascular disease risk evaluation. Journal of Applied Computer Science & Mathematics, 2009. 5 (3), 29-32.
8) Syed Umar Amin, Kavita Agarwal, Dr. Rizwan Beg. Data Mining in Clinical Decision Support Systems for Diagnosis, Prediction and Treatment of Heart Disease. International Journal of Advanced Research in Computer Engineering & Technology (IJARCET). 2013. 2(1). 218-223.
9) Murphy C. K. Identifying diagnostic errors with induced decision trees. Medical Decision Making, 2001. 21(5), 368-375.
10) Qu Y., Adam B. L., Yasui Y., Ward M. D., Cazares L. H., Schellhammer P. F., et al. Boosted decision tree analysis of surface-enhanced laser desorption/ionization mass spectral serum profiles discriminates prostate cancer from noncancer patients. Clinical Chemistry, 2002. 48(10), 1835-1843.
11) Won Y., Song H., Kang T. W., Kim J., Han B., Lee S. Pattern analysis of serum proteome distinguishes renal cell carcinoma from other urologic diseases and healthy persons. Proteomics, 2003. 3(12), 2310-2316.
12) Geurts P., Fillet M., de Seny D., Meuwis M. A., Malaise M., Merville M. P., et al. Proteomic mass spectra classification using decision tree based ensemble methods. Bioinformatics, 2005. 21(14), 3138-3145.
13) Balla J. I., Iansek R., Elstein A. Bayesian diagnosis in presence of preexisting disease. Lancet. 1985. 325 (8424), 326-329.
14) Harris N. L. Probabilistic belief networks for genetic counseling. Computer Methods and Programs in Biomedicine, 1990. 32(1), 37-44.
15) Sadeghi S., Barzi A., Sadeghi N., King B. A Bayesian model for triage decision support. International Journal of Medical Informatics, 2006. 75(5), 403-411.
16) Baxt W. G. Application of artificial neural networks to clinical medicine. Lancet, 1995. 346 (8983), 1135-1138.
17) Chae Y. M., Park K. E., Park K. S., Bae M. Y. Development of medical decision support system for Leukemia management. Expert Systems with Applications. 1998. 15, 309-315.
18) West D., West V. Model selection for a medical diagnostic decision support system: A breast cancer detection case. Artificial Intelligence in Medicine. 2000. 20 (3), 183-204.
19) Veropoulos K., Cristianini N., Campbell C. The application of support vector machines to medical decision support: A Case Study. In Proceedings of the ECCAI advanced course on artificial intelligence (ACAI 1999), Jul. 5-16, 1999, Chania, Greece.
20) Schubert F., Muller J., Fritz B., Lichter P., Eils R. Understanding the classification of tumors with a support vector machine: A case-based explanation scheme. Proceedings of the German conference on bioinformatics (GCB 2003), Neuherberg/Garching, Oct. 12-14, 123-127.
21) Prados J., Kalousis A., Sanchez J. C., Allard L., Carrette O., Hilario M. Mining mass spectra for diagnosis and biomarker discovery of cerebral accidents. Proteomics, (2004). 4 (8), 2320-2332.
22) Department of Health. Chronic Diseases and Conditions. New York State. May 201. August 2015.
23) Wang J, Gao J, Yao H, Wu Z, Wang M, Qi J. Diagnostic accuracy of serum HE4, CA125 and ROMA in patients with ovarian cancer: a meta-analysis. Tumour Biol. (2014); 35 (6): 6127-38.
24) CAS Differential Diagnostic checklist. Available at http://www.communicationstationspeech.com/cas-differential-diagnostic-checklist/

### SELECTED EMBODIMENTS

Although the above description and the attached claims disclose a number of embodiments of the present invention, other alternative aspects of the invention are disclosed in the following further embodiments.

### Embodiment 1

A method for fabricating a nucleic acid chip comprising a plurality of distinct single stranded nucleic acids at spatially discrete locations on a solid substrate, the method comprising: reacting samples of at least one of urine or saliva from patients with a pool of randomly generated aptamers; separating aptamers that bind to biomarkers in the samples from the patients from the aptamers that do not bind; determining the sequences of at least a portion of the aptamers that bind to the biomarkers to develop a library of distinct aptamers that each bind to different biomarkers in the samples; immobilizing single stranded nucleic acids with sequences that are complementary to one aptamer in the library of aptamers at each discrete location on the solid substrate.

### Embodiment 2

The method of claim 1, wherein the step of reacting the samples with the aptamers further comprises: applying the samples to a membrane; and applying the pool of randomly generated aptamers to the membrane to allow the aptamers to form complexes with the biomarkers in the samples.

### Embodiment 3

The method of claim 2, wherein the step of separating the aptamers further comprises washing the unbound aptamers off of the membrane with a buffer solution.

### Embodiment 4

A method for fabricating a nucleic acid chip comprising a plurality of distinct single stranded nucleic acids at spatially discrete location on a solid substrate, the method comprising: reacting samples of a bodily fluid comprising at least one of blood, urine, or saliva from patients with and without at least one chronic disease with a pool of randomly generated aptamers; separating aptamers that bind to biomarkers in the samples from the patients from the aptamers that do not bind; determining the sequences of at least a portion of the aptamers that bind to the biomarkers to develop a library of distinct aptamers that each bind to different biomarkers in the samples; and immobilizing single stranded nucleic acids with sequences that are complementary to one aptamer in the library of aptamers at each discrete location on the solid substrate.

### Embodiment 5

A biochip for diagnosing a chronic disease, the biochip comprising: a solid substrate; an array of spots at spatially discrete locations on the solid substrate, each spot in the array comprising single stranded nucleic acids immobilized on the solid substrate that have a sequence complementary to one aptamer in a set of aptamers, each aptamer in the set of aptamers having a unique sequence, the array of spots including at least one spot for each aptamer in the set of aptamers, the set of aptamers identified as binding to biomarkers in a pool of sample types from subjects, the sample types comprising both samples from subjects with and without at least one chronic disease.

### Embodiment 6

The biochip of claim 4, wherein the at least one chronic disease comprises at least two chronic diseases.

### Embodiment 7

The biochip of claim 4, wherein the set of aptamers are subset of all of the aptamers that bound to biomarkers in the pool of sample types identified as having the highest affinity for their respective biomarkers.

### Embodiment 8

The biochip of claim 4, wherein the set of aptamers comprises at least 50 aptamers.

### Embodiment 9

The biochip of claim 4, wherein the set of aptamers comprises at least 100 aptamers.

### Embodiment 10

The biochip of claim 4, wherein the set of aptamers comprises at least 1,000 aptamers.

### Embodiment 11

The biochip of claim 4, wherein the set of aptamers comprises 5,000 aptamers.

### Embodiment 12

The biochip of claim 10, wherein the at least one chronic disease comprises at least 30 chronic diseases.

### Embodiment 13

A biochip for diagnosing a chronic disease, the biochip comprising: a solid substrate; and an array of spots at spatially discrete locations on the solid substrate, each spot in the array comprising a plurality of single stranded nucleic acids immobilized on the solid substrate that have a sequence complementary to one aptamer of a set of aptamers, each aptamer in the set of aptamers having a unique sequence, the array of spots including at least one spot for each aptamer in the set of aptamers, the set of aptamers identified as binding to a biomarker in a pool of sample types comprising at least one of urine and saliva.

### Embodiment 14

The biochip of claim 6, wherein the pool of sample types comprises urine, saliva, and blood.

### Embodiment 15

The biochip of claim 6, wherein the pool of sample types comprises urine, and saliva.

### Embodiment 16

The biochip of claim 6, wherein the pool of sample types comprises urine, and blood.

### Embodiment 17

The biochip of claim 6, wherein the pool of sample types comprises saliva, and blood.

### Embodiment 18

The biochip of claim 6, wherein pool of sample types comprises samples from subjects with and without chronic diseases.

### Embodiment 19

A kit for diagnosing chronic diseases, the kit comprising: at least one of the biochips of claim 5, a sufficient quantity of the set of aptamers of claim 2 in a sealed container for use of all of the plurality of biochips of claim 5.

### Embodiment 20

The kit of claim 19, further comprising: blotting membrane; washing buffer binding buffer; blocking buffer; and standard control solution.

### Embodiment 21

A method of detecting a chronic disease, the method comprising: reacting a sample from a patient with unknown disease status with a set of aptamers to form biomarker-aptamer complexes; separating the complexes from the unbound aptamers; amplifying and labeling the biomarker binding aptamers in the complexes to produce labeled aptamers; reacting the labeled aptamers with physically separate pools of single stranded nucleic acids to hybridize the labeled aptamers with the single stranded nucleic acids, wherein each pool contains aptamers with sequences that are complementary to only one aptamer in the set of aptamers; separating the hybridized labeled aptamers from the non-hybridized labeled aptamers; detecting an optical quality emitted by the hybridized labeled aptamers for each separate pool; processing each optical quality detected for each separate pool as input data for a DNN based algorithm to determine whether the patient has the chronic disease; and outputting an indication of whether the patient has the chronic disease based on the determination.

### Embodiment 22

The method of claim 21, wherein the step of processing each optical quality as input data further comprising inputting the data directly as image data into a CNN based DNN algorithm.

### Embodiment 23

The method of claim 21, wherein the step of processing each optical quality as input data further comprising: determining an intensity for each optical quality; and inputting the intensity into an RBM based DNN algorithm.

### Embodiment 24

The method of claim 22, wherein the step of processing each optical quality as input data further comprising: determining an intensity for each optical quality; inputting the intensity into an RBM based DNN algorithm; and merging the outputs of the RBM and CNN based DNN algorithm to determine whether the patient has a chronic disease.

### Embodiment 25

The method of claim 21, wherein the sample is a body fluid, urine, saliva, cheek swab, mucus; whole blood, blood, serum, plasma, semen, lymph, fecal extract, or sputum, or a combination thereof.

### Embodiment 26

The method of claim 21, wherein the step of reacting the sample with a set of aptamers is performed on a membrane.

### Embodiment 27

The method of claim 21, wherein the biomarker binding aptamers are labeled with a fluorescent dye.

### Embodiment 28

The method of claim 27, wherein the optical quality is a fluorescence of the fluorescent dye.

### Embodiment 29

The method of claim 28, wherein the single stranded nucleic acids are immobilized in spatially separate pools in an array on a solid substrate.

### Embodiment 30

A method for diagnosing a chronic disease with a clinical decision support system operating on a server, the method comprising: receiving, by the clinical decision support system operating on the server, image data referenced to an identifier of a sample, the image data representing detected radiation emitted from labeled aptamers that formed complexes with biomarkers in the sample; processing the image data by the clinical decision support system with a DNN based classifier to determine whether or not the sample is from a patient with a chronic disease; and outputting, by the clinical decision support system, an indication of whether or not the sample is from a patient with a chronic disease.

### Embodiment 31

The method of claim 30, wherein outputting of the indication is sent to a computer connected on a remote device and displayed on a display.

### Embodiment 32

The method of claim 30, wherein the processing of the image data by the clinical decision support system comprises determining a data value for the intensity of the image data and inputting those data values into an RBM based DNN classifier.

### Embodiment 33

The method of claim 30, wherein the processing of the image data by the clinical decision support system further comprises inputting the image directly into a CNN based DNN classifier.

### Embodiment 34

The method of claim 32, wherein the processing of the image data by the clinical decision support system further comprises inputting the image directly into a CNN based DNN classifier.

### Embodiment 35

The method of claim 34, wherein the processing of the image data by the clinical decision support system further comprises combining the outputs of the CNN and RBM based classifier to determine whether or not the patient has the chronic disease.

### Embodiment 36

A method for training a DNN based classifier for diagnosing a chronic disease using image data relating to concentrations of biomarkers, the method comprising: reacting samples from patients with the chronic disease and without the chronic disease with a set of aptamers to form biomarker-aptamer complexes; separating the complexes from the unbound aptamers; amplifying and labeling the biomarker binding aptamers in the complexes to produce labeled aptamers; reacting the labeled aptamers with physically separate pools of single stranded nucleic acids to hybridize the labeled aptamers with the single stranded nucleic acids, wherein each pool contains aptamers with sequences that are complementary to only one aptamer in the set of aptamers; separating the hybridized labeled aptamers from the non-hybridized labeled aptamers; detecting an optical quality emitted by the hybridized labeled aptamers for each separate pool; processing each optical quality detected for each separate pool as input data to train a DNN based classifier; and repeating steps (a)-(g) until the DNN based classifier can diagnose an unknown sample for the chronic disease with sufficient sensitivity and specificity.

### Embodiment 37

A system, comprising: a set of aptamers configured for forming biomarker-aptamer complexes with biomarkers in a biological sample from a patient with unknown status for a chronic disease; a reaction module reaction configured for allowing the set of aptamers to react with the biological sample; a means for spearing biomarker-bound aptamers from unbound aptamers; an amplifying and labeling module configured for amplifying and labeling the biomarker-bound biomarker to produce labeled aptamers; a biochip of claim 8 or 16 configured for hybridizing with the labeled aptamers; a means for separating biochip-hybridized labeled aptamers from non-hybridized labeled aptamers; a means for detecting optical qualities emitted by the biochip-hybridized labeled aptamers to generate an image data; a computer configured for processing the image data using DNN based algorithm to determine whether the patient has the chronic disease; and an output module configured for outputting an indication of whether the patient has the chronic disease based on the determination.

### Embodiment 38

The system of claim 41, wherein the biological sample is a body fluid, urine, saliva, cheek swab, mucus; whole blood, blood, serum, plasma, semen, lymph, fecal extract, or sputum, or a combination thereof.

### Embodiment 39

The system of claim 41, further comprising the biological sample as a part of the system.

### Embodiment 40

The system of claim 41, wherein the computer comprises: a memory configured for storing one or more programs; and one and more processors configured for executing the one or more programs, wherein the one or more programs comprise instructions for operating the system and/or modules thereof.

### Embodiment 41

The system of claim 41, wherein the computer comprises one or more of a server, a network, and a database.

### Embodiment 42

The system of claim 41, further comprising a treatment module configured for treating a patient determined as having the chronic disease.

### Embodiment 43

The system of claim 45, wherein the treatment module comprises a therapeutic agent for cancer, cardiovascular diseases, chronic respiratory diseases, diabetes, lupus, or stroke.

### Embodiment 44

A method of detecting or diagnosing a chronic disease in a subject, comprising: obtaining a biological sample from the subject; providing a system of claim 41; and operating the system to analyze the biological sample, thereby detecting or diagnosing the chronic disease in the subject.

### Embodiment 45

A method of treating a subject with unknown status for a chronic disease, comprising: obtaining a biological sample from the subject; providing a system of claim 41; operating the system to analyze the biological sample, thereby detecting or diagnosing the chronic disease in the subject; providing a therapeutic agent for the chronic disease; and administering the therapeutic agent to the subject, thereby treating the subject.

### Embodiment 46

A method of treating a chronic disease, comprising: providing a subject diagnosed with the chronic disease using the method of claim 26, 34, or 47; providing a therapeutic agent for the chronic disease; and administering the therapeutic agent to the subject, thereby treating the subject.

### CONCLUSIONS

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A biochip for diagnosing a chronic disease, the biochip comprising:
a solid substrate;
an array of spots at spatially discrete locations on the solid substrate, each spot in the array comprising single stranded nucleic acids immobilized on the solid substrate that have a sequence complementary to one aptamer in a set of aptamers, each aptamer in the set of aptamers having a unique sequence, the array of spots including at least one spot for each aptamer in the set of aptamers, the set of aptamers identified as binding to biomarkers in a pool of sample types from subjects, the sample types comprising both samples from subjects with and without at least one chronic disease.

2. The biochip of claim 1, wherein the at least one chronic disease comprises at least two chronic diseases.

3. The biochip of claim 1, wherein the set of aptamers are subset of all of the aptamers that bound to biomarkers in the pool of sample types identified as having the highest affinity for their respective biomarkers.

4. The biochip of claim 1, wherein the set of aptamers comprises at least 50 aptamers.

5. The biochip of claim 1, wherein the set of aptamers comprises at least 100 aptamers.

6. The biochip of claim 1, wherein the set of aptamers comprises at least 1,000 aptamers.

7. The biochip of claim 1, wherein the set of aptamers comprises 5,000 aptamers.

8. The biochip of claim 6, wherein the at least one chronic disease comprises at least 30 chronic diseases.

9. A biochip for diagnosing a chronic disease, the biochip comprising:
a solid substrate; and
an array of spots at spatially discrete locations on the solid substrate, each spot in the array comprising a plurality of single stranded nucleic acids immobilized on the solid substrate that have a sequence complementary to one aptamer of a set of aptamers, each aptamer in the set of aptamers having a unique sequence, the array of spots including at least one spot for each aptamer in the set of aptamers, the set of aptamers identified as binding to a biomarker in a pool of sample types comprising at least one of urine and saliva.

10. The biochip of claim 2, wherein the pool of sample types comprises urine, saliva, and blood.

11. The biochip of claim 2, wherein the pool of sample types comprises urine, and saliva.

12. The biochip of claim 2, wherein the pool of sample types comprises urine, and blood.

13. The biochip of claim 2, wherein the pool of sample types comprises saliva, and blood.

14. The biochip of claim 2, wherein pool of sample types comprises samples from subjects with and without chronic diseases.

15. A kit for diagnosing chronic diseases, the kit comprising:
at least one of the biochips of claim 5,
a sufficient quantity of the set of aptamers of claim 2 in a sealed container for use of all of the plurality of biochips of claim 5.

16. The kit of claim 15, further comprising:
blotting membrane;
washing buffer
binding buffer;
blocking buffer; and
standard control solution.

17. A method of detecting a chronic disease, the method comprising:
(a) reacting a sample from a patient with unknown disease status with a set of aptamers to form biomarker-aptamer complexes;
(b) separating the complexes from the unbound aptamers;
(c) amplifying and labeling the biomarker binding aptamers in the complexes to produce labeled aptamers;
(d) reacting the labeled aptamers with physically separate pools of single stranded nucleic acids to hybridize the labeled aptamers with the single stranded nucleic acids, wherein each pool contains aptamers with sequences that are complementary to only one aptamer in the set of aptamers;
(e) separating the hybridized labeled aptamers from the non-hybridized labeled aptamers;
(f) detecting an optical quality emitted by the hybridized labeled aptamers for each separate pool;
(g) processing each optical quality detected for each separate pool as input data for a DNN based algorithm to determine whether the patient has the chronic disease; and
(h) outputting an indication of whether the patient has the chronic disease based on the determination.

18. The method of claim 17, wherein the step of processing each optical quality as input data further comprising inputting the data directly as image data into a CNN based DNN algorithm.

19. The method of claim 17, wherein the step of processing each optical quality as input data further comprising:
determining an intensity for each optical quality; and
inputting the intensity into an RBM based DNN algorithm.

20. The method of claim 18, wherein the step of processing each optical quality as input data further comprising:
determining an intensity for each optical quality;
inputting the intensity into an RBM based DNN algorithm; and
merging the outputs of the RBM and CNN based DNN algorithm to determine whether the patient has a chronic disease.

21. The method of claim 17, wherein the sample is a body fluid, urine, saliva, cheek swab, mucus; whole blood, blood, serum, plasma, semen, lymph, fecal extract, or sputum, or a combination thereof.

22. The method of claim 17, wherein the step of reacting the sample with a set of aptamers is performed on a membrane.

23. The method of claim 17, wherein the biomarker binding aptamers are labeled with a fluorescent dye.

24. The method of claim 23, wherein the optical quality is a fluorescence of the fluorescent dye.

25. The method of claim 24, wherein the single stranded nucleic acids are immobilized in spatially separate pools in an array on a solid substrate.

26. A method for diagnosing a chronic disease with a clinical decision support system operating on a server, the method comprising:
receiving, by the clinical decision support system operating on the server, image data referenced to an identifier of a sample, the image data representing detected radiation emitted from labeled aptamers that formed complexes with biomarkers in the sample;
processing the image data by the clinical decision support system with a DNN based classifier to determine whether or not the sample is from a patient with a chronic disease; and
outputting, by the clinical decision support system, an indication of whether or not the sample is from a patient with a chronic disease.

27. The method of claim 26, wherein outputting of the indication is sent to a computer connected on a remote device and displayed on a display.

28. The method of claim 26, wherein the processing of the image data by the clinical decision support system comprises determining a data value for the intensity of the image data and inputting those data values into an RBM based DNN classifier.

29. The method of claim 26, wherein the processing of the image data by the clinical decision support system further comprises inputting the image directly into a CNN based DNN classifier.

30. The method of claim 28, wherein the processing of the image data by the clinical decision support system further comprises inputting the image directly into a CNN based DNN classifier.

31. The method of claim 30, wherein the processing of the image data by the clinical decision support system further comprises combining the outputs of the CNN and RBM based classifier to determine whether or not the patient has the chronic disease.
